(19) United States
(12) Patent Application Publication
(10) Pub. No.: US 2009/0149429 A1
(43) Pub. Date: Jun. 11, 2009

(54) ANTIVIRAL COMPOUNDS

(75) Inventors: Esther Arranz Plaza, Madrid (ES); Kraig M. Yager, Salt Lake City, UT (US); David Allen Gerrish, Salt Lake City, UT (US); Mark B. Anderson, Salt Lake City, UT (US); In Chul Kim, Sandy, UT (US); Dange Vijay Kumar, Salt Lake City, UT (US)

Correspondence Address: MYRIAD GENETICS INC.;INTELLECUTAL PROPERTY DEPARTMENT, 320 WAKARA WAY, SALT LAKE CITY, UT, 84108, US

(73) Assignee: Myriad Genetics, Incorporated, Salt Lake City, UT (US)

(21) Appl. No.: 11/993,519

(22) PCT Filed: Jun. 22, 2006
(86) PCT No.: PCT/US06/24493

§ 371 Date: Dec. 29, 2008

Related U.S. Application Data
(60) Provisional application No. 60/692,826, filed on Jun. 22, 2005.
(60) Provisional application No. 60/765,790, filed on Feb. 7, 2006.

(51) Int. Cl. A61K 31/56; C07J 53/00; A61K 31/58; A61P 31/12; A61P 31/18

(52) U.S. Cl. 514/171; 552/511; 514/182; 514/172; 514/176

(57) ABSTRACT

The invention relates to compounds, pharmaceutical compositions and methods useful for treating viral infection.

### FIELD OF THE INVENTION

The present invention relates generally to methods, compounds, and pharmaceutical compositions for treating (and delaying the onset of) viral infection. The compositions and methods useful in the treatment of viral infections caused by viruses such as HIV, hepatitis B virus, hepatitis C virus, herpes simplex virus type-1, herpes simplex virus type-2, herpes simplex virus type-4 (Epstein-Barr virus), influenza viruses, smallpox viruses, coronaviruses (i.e., SARS-associated), and West Nile virus.

### BACKGROUND OF THE INVENTION

Viral infection of humans is a major health problem, and viral infection of domesticated animals is a major economic concern. Combating viral infection has proven to be highly effective in some cases like smallpox where the disease was essentially eradicated with the advent of smallpox vaccination. Although smallpox was essentially eradicated by about 1980, there is considerable justified fear of the emergence of a new epidemic of smallpox since there are existing stockpiles of the virus and bioterrorism has moved beyond the realm of possibility to reality. Other viral infections have been much more difficult to fight. Hepatitis B and C, human immunodeficiency virus (HIV), herpes simplex viruses, and influenza are just a few prominent members of a list of viruses that pose significant health threats worldwide. Additionally, emerging viral infections continue to threaten the world with human epidemics, as is illustrated by the recent outbreak of severe acute respiratory syndrome (SARS) which has now been associated with coronavirus infection. Treatments currently available for many viral infections are often associated with adverse side effects. In addition, antiviral therapeutics directed towards specific viral gene products frequently have the effect of driving the selection of viruses resistant to such therapeutics, and viral strains resistant to current methods of treatment are an increasing problem. Accordingly, there is a clear and ever-present need for new antiviral treatments.

### BRIEF SUMMARY OF THE INVENTION

The present invention generally relates to compounds and methods for treating viral infections. In addition, the present invention also relates to treating and/or delaying the onset of symptoms caused by viral infections. The invention provides compounds of Formulae I-III and pharmaceutical compositions having one or more compounds of Formulae I-III and one or more pharmaceutically acceptable excipients. Compounds of Formula I-III include:

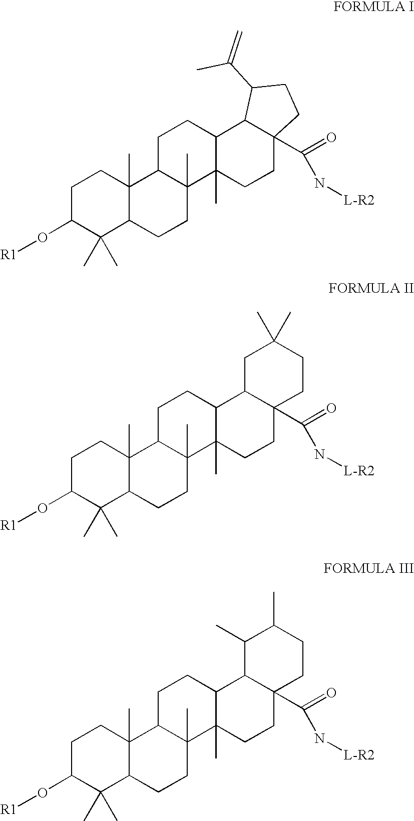

where L is an alkyl group having from 1 to 10 carbons which can be saturated or partially saturated; one or more of the carbons of the alkyl group of L can be substituted with —O—, —S—, —N—, —C(═O)—, —NC(═O)—, —C(═O)N—, —SO₂, —NSO₂, —SO₂N—, and —NC(═O)N—; L can be substituted with one or more substituents chosen from hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;
R1 is chosen from hydro, —C(═O)—(CH₂)ₘ—CH₃, —C(═O)—(CH₂)ₘ—C(CH₃)₂—COOH;
R2 is chosen from cycloalkyl, aryl, heterocycle, and heteroaryl, optionally substituted with one or more substituents chosen from hydro, hydroxyl, halo, alkyl, alkoxy, alkylthio, arylthio, thiocarbonyl, O-carboxy, C-carboxy, O-carbamyl, O-thiocarbamyl, N-carbamyl, N-thiocarbamyl, ester, haloalkyl, haloalkoxy, cycloalkyl, aryl, heteroaryl, heterocycle, —C(═O)OH, —CH(CH₃)C(═O)OH; —CH₂C(═O)OH, —C(CH₃)₂C(═O)OH, —C(CH₃)(CH₂CH₃)C(═O)OH, —CH(CH₂CH₃)C(═O)OH, —CH═C(CH₃)C(═O)OH, —C(CH₂CH₃)₂C(═O)OH, —N(C₁₋₃ alkyl)₂, —NH(C₁₋₃ alkyl), —C(—O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;
n is an integer chosen from 0, 1, 2, and 3; and
m is an integer chosen from 0-10.

Thus, in a related aspect, the present invention provides methods for treating viral infection by administering to a patient in need of such treatment a pharmaceutical composition or medicament having a therapeutically (or prophylactically) effective amount of a compound of Formulae I-III.

In another aspect of the invention, methods for inhibiting viral maturation are also provided by administering to a patient in need of such treatment a pharmaceutical composition or medicament having an amount of a compound of Formulae I-III sufficient to inhibit the maturation of a virus from human or animal cells. In one particular aspect of this embodiment of the invention, the method of inhibiting viral maturation involves treating humans infected with a virus with a compound of Formulae I-III.

In addition, the present invention further provides methods for delaying the onset of viral infection symptoms comprising administering a pharmaceutical composition or medicament having a prophylactically effective amount of a compound of Formulae I-III to an individual having a viral infection, or at risk of infection by a virus, or at risk of developing symptoms of viral infection. In one particular aspect of this embodiment of the invention, the method of inhibiting or delaying the onset of viral infection symptoms involves treating humans infected with a virus with a compound of Formulae I-III.

In one aspect of the invention, a method is provided for treating a person who is a carrier of any of the HIV family of retroviruses, i.e., infected with HIV, but has not developed AIDS (which is defined by more serious AIDS-defining illnesses and/or a decline in the circulating CD4 cell count to below a level that is compatible with effective immune function). The method includes identifying such an individual in need of treatment and administering to the individual a pharmaceutical composition or medicament having a therapeutically effective amount of a compound of Formula I-III. Thus, the method can be used in treating acute primary HIV infection syndrome (which can be asymptomatic or associated with an influenza-like illness with fevers, malaise, diarrhea and neurologic symptoms such as headache) or asymptomatic infection (which is the long latent period with a gradual decline in the number of circulating CD4 T-cells).

In another aspect, a method is provided for treating a person who is either actively infected with Hepatitis B virus (HBV), Hepatitis C virus (HCV), or who is a carrier of these viruses that has either not developed symptoms of the viral infection (which is defined by liver damage) or has experienced diminution of such symptoms, or who has recently been exposed to such viruses. The method includes identifying such an individual in need of treatment and administering to the individual a pharmaceutical composition or medicament having a therapeutically effective, or prophylactically effective, amount of a compound of Formulae I-III.

In another aspect, a method is provided for treating a person who is either actively infected with herpes simplex virus type-1, type-2, or type-4 (also known as Epstein-Barr virus), or who is a carrier of these viruses who has either not developed symptoms of the viral infection or has experienced diminution of such symptoms, or who has recently been exposed to such viruses. The method includes identifying such an individual in need of treatment and administering to the individual a pharmaceutical composition or medicament having a therapeutically effective, or prophylactically effective, amount of a compound of Formulae I-III.

In another aspect, a method is provided for treating an individual who is either actively infected with influenza virus type-A, type-B, or type-C, or who is a carrier of these viruses who has either not developed symptoms of the viral infection, or has experienced diminution of such symptoms, or who has recently been exposed to such viruses. The method includes identifying such an individual in need of treatment and administering to the individual a pharmaceutical composition or medicament having a therapeutically effective, or prophylactically effective, amount of a compound of Formulae I-III.

In yet another aspect, a method is provided for treating a person who is either actively infected with any of the pox virus family of viruses, i.e., the smallpox virus, or who is a carrier of these viruses who has either not developed symptoms of the viral infection (which is defined by more serious smallpox-defining illnesses) or has experienced diminution of such symptoms, or who has recently been exposed to such viruses. The method includes identifying such an individual in need of treatment and administering to the individual a pharmaceutical composition or medicament having a therapeutically effective, or a prophylactically effective, amount of a compound of Formulae I-III.

In another aspect, a method is provided for treating a person who is either actively infected with any of the coronavirus family of viruses, i.e., infected with a SARS-associated coronavirus, or who is a carrier of such viruses who has either not developed symptoms of the viral infection (which is defined by more serious SARS-defining illnesses) or who has experienced diminution of such symptoms, or who has recently been exposed to such viruses. The method includes identifying such an individual in need of treatment and administering to the individual a pharmaceutical composition or medicament having a prophylactically effective amount of a compound of Formulae I-III.

In yet another aspect, a method is provided for treating a person or an animal that is either actively infected with West Nile virus, or is a carrier of the West Nile virus and has either not developed symptoms of the viral infection, or has experienced diminution of such symptoms, or has recently been exposed to West Nile virus. The method includes identifying such an individual in need of treatment and administering to the individual a pharmaceutical composition or medicament having a prophylactically effective amount of a compound of Formulae I-III.

The compounds of Formulae I-III for use in the instant invention can be provided as a pharmaceutical composition with one or more salts, carriers, or excipients. Some of the compounds for use in the invention have chiral centers, and the invention therefore includes the use of all stereoisomers, enantiomers, diastereomers, and mixtures thereof.

The present invention also provides pharmaceutical compositions or medicaments for the combination therapy of viral infections. The compositions comprise a therapeutically effective amount of a first compound according to Formula I-III and a therapeutically effective amount of a second antiviral compound, which is different from the first compound. Examples of antiviral compounds include, but are not limited to, protease inhibitors, nucleoside reverse transcriptase inhibitors, non-nucleoside reverse transcriptase inhibitors, integrase inhibitors, fusion inhibitors, immunomodulators, and vaccines.

The compounds of the invention can be used to treat a variety of additional disease or conditions such as hypertension, cancer (including metastasis), immune system related diseases, autoimmune diseases, bacterial infections (e.g., those of the digestive track), retinopathies, and neurological disorders.

The foregoing and other advantages and features of the invention, and the manner in which they are accomplished, will become more readily apparent upon consideration of the following detailed description of the invention taken in conjunction with the accompanying examples, which illustrate preferred and exemplary embodiments.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION OF THE INVENTION

The invention provides compounds of Formulae I-III, which are useful for treating viral infections and symptoms thereof. Compounds of Formulae I-III include:

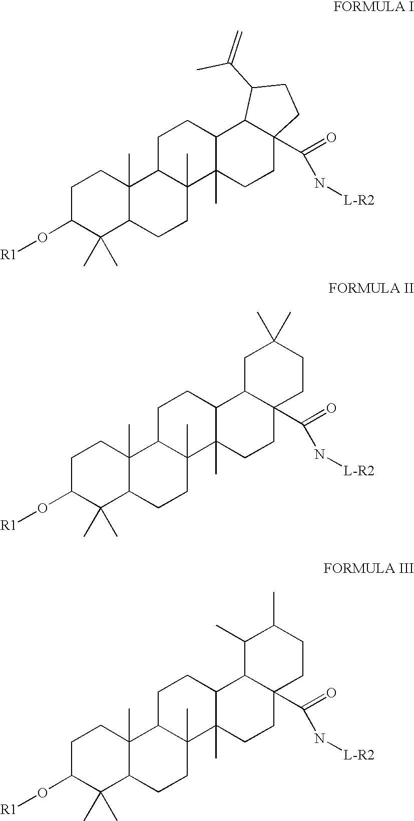

where L is an alkyl group having from 1 to 10 carbons which can be saturated or partially saturated; one or more of the carbons of the alkyl group of L can be substituted with —O—, —S—, —N—, —C(═O)—, —NC(═O)—, —C(═O)N—, —SO₂, —NSO₂, —SO₂N—, and —NC(═O)N—; L can have one or more substituents chosen from hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;
R1 is chosen from hydro, —C(═O)—(CH₂)ₘ—CH₃, —C(═O)—(CH₂)ₘ—C(CH₃)₂—COOH;
R2 is chosen from a cycloalkyl, aryl, heterocycle, and heteroaryl ring optionally substituted with one or more substituents chosen from hydro, hydroxyl, halo, alkyl, alkoxy, alkylthio, arylthio, thiocarbonyl, O-carboxy, C-carboxy, O-carbamyl, O-thiocarbamyl, N-carbamyl, N-thiocarbamyl, ester, haloalkyl, haloalkoxy, cycloalkyl, aryl, heteroaryl, heterocycle, —C(═O)OH, —CH(CH₃)C(═O)OH; —CH₂C(═O)OH, —C(CH₃)₂C(═O)OH, —C(CH₃)(CH₂CH₃)C(═O)OH, —CH(CH₂CH₃)C(═O)OH, —CH═C(CH₃)C(═O)OH, —C(CH₂CH₃)₂C(═O)OH, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;
n is an integer chosen from 0, 1, 2, and 3;
m is an integer chosen from 1-10; and pharmaceutically acceptable salts thereof.

In one embodiment, the invention provides compounds of Formula I(a)-III(a)

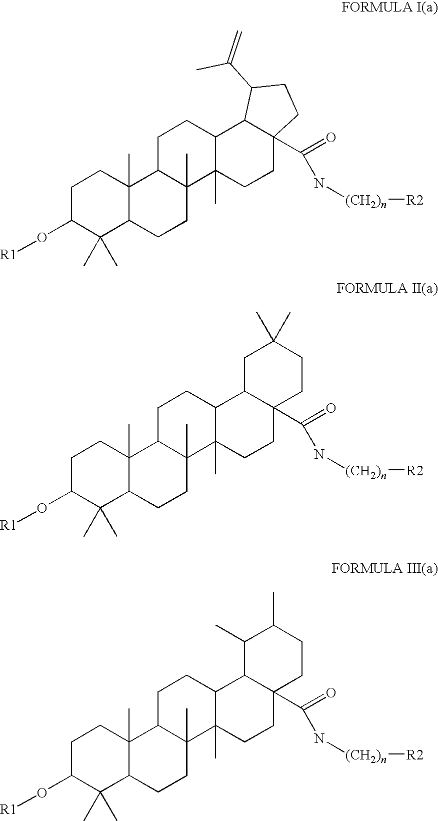

where R1 is —C(═O)—CH₂—C(CH₃)₂—COOH;
R2 is chosen from a cycloalkyl, aryl, heterocycle, and heteroaryl ring optionally substituted with one or more substituents chosen from hydro, hydroxyl, halo, alkyl, alkoxy, alkylthio, arylthio, thiocarbonyl, O-carboxy, C-carboxy, O-carbamyl, O-thiocarbamyl, N-carbamyl, N-thiocarbamyl, ester, haloalkyl, haloalkoxy, cycloalkyl, aryl, heteroaryl, heterocycle, —C(═O)OH, —CH(CH₃)C(═O)OH; —CH₂C(═O)OH, —C(CH₃)₂C(═O)OH, —C(CH₃)(CH₂CH₃)C(═O)OH, —CH(CH₂CH₃)C(═O)OH, —CH═C(CH₃)C(═O)OH, —C(CH₂CH₃)₂C(═O)OH, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;
n is an integer chosen from 0, 1, 2, and 3; and pharmaceutically acceptable salts thereof.

The compounds of Formulae I (including I(a)) include those having the following core structure:

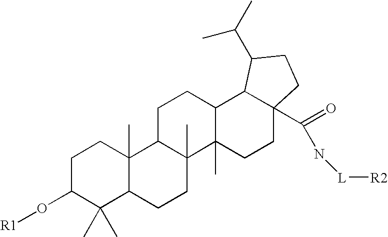

and pharmaceutically acceptable salts thereof, wherein R1, R2 and L are as defined above.

In one embodiment, the invention provides compounds of Formulae I(a) and pharmaceutical compositions comprising the compound and one or more pharmaceutically acceptable excipients, wherein R1 is —C(═O)—CH₂—C(CH₃)₂—COOH; R2 is a phenyl group substituted with one or more substituents chosen from hydroxyl, halo, alkyl, alkoxy, alkylthio, arylthio, thiocarbonyl, O-carboxy, C-carboxy, O-carbamyl, O-thiocarbamyl, N-carbamyl, N-thiocarbamyl, ester, haloalkyl, haloalkoxy, cycloalkyl, aryl, heteroaryl, heterocycle, —C(═O)OH, —CH(CH₃)C(═O)OH; —CH₂C(═O)OH, —C(CH₃)₂C(═O)OH, —C(CH₃)(CH₂CH₃)C(—O)OH, —CH(CH₂CH₃)C(═O)OH, —CH═C(CH₃)C(═O)OH, —C(CH₂CH₃)₂C(═O)OH, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂; and n is an integer chosen from 0, 1, 2, and 3. In one aspect of this embodiment, the stereochemistry of the core betulin moiety is preserved.

A pharmaceutically acceptable salt of the compound of the present invention is exemplified by a salt with an inorganic acid such as hydrochloric acid, hydrobromic acid, sulfuric acid, phosphoric acid, nitric acid and the like, and a salt with an organic acid such as acetic acid, propionic acid, succinic acid, maleic acid, fumaric acid, benzoic acid, citric acid, malic acid, methanesulfonic acid, benzenesulfonic acid and the like. Their hydrates (1 hydrate, 2 hydrate, 3 hydrate, ½ hydrate, 3/2 hydrate, ¼ hydrate, ⅘ hydrate, ⅕ hydrate, ¾ hydrate, ⅓ hydrate, 5/3 hydrate, 5/4 hydrate etc.), solvates and the like are also encompassed in the compound of the present invention. In addition, N-oxide compounds are also encompassed in the compound of the present invention.

In addition, pharmaceutically acceptable salts include acid salt of inorganic bases, such as salts containing alkaline cations (e.g., Li+, Na+ or K+), alkaline earth cations (e.g., Mg++, Ca++ or Ba++), the ammonium cation, as well as acid salts of organic bases, including aliphatic and aromatic substituted ammonium, and quaternary ammonium cations, such as those arising from protonation of peralkylation of triethylamine, N,N-diethylamine, N,N-dicyclohexylamine, pyridine, N,N-dimethylaminopyridine (DMAP), 1,4-diazabiclo[2.2.2]octane (DABCO), 1,5-diazavicyclo[4.3.0]non-5-ene (DBN) and 1,8-diazabicyclo[5.4.0]undec-7-ene (DBU).

Additionally, the compounds of Formulae I-III can contain asymmetric carbon atoms and can therefore exist in racemic and optically active forms. Thus, optical isomers or enantiomers, racemates, and diastereomers are also encompassed in the compounds of Formulae I-III. The methods of present invention include the use of all such isomers and mixtures thereof. Methods of separation of enantiomeric and diastereomeric mixtures are well known to one skilled in the art. The present invention encompasses any isolated racemic or optically active form of compounds described in Formulae I-III, or any mixture thereof, which possesses anti-viral activity.

In one embodiment of the invention, the stereochemistry of the compounds of Formulae I-III is equivalent to that of the natural product from which the compound was derived (e.g., betulinic acid).

Unless specifically stated otherwise or indicated by a bond symbol (dash or double dash), the connecting point to a recited group will be on the right-most stated group. Thus, for example, a hydroxyalkyl group is connected to the main structure through the alkyl and the hydroxyl is a substituent on the alkyl.

As used herein, the term “alkyl” refers to a saturated aliphatic hydrocarbon including straight chain and branched chain groups. Preferably, the alkyl group has 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc. up to and including 20 carbon atoms). More preferably, it is a medium size alkyl having 1 to 10 carbon atoms. Even more preferably, it is a lower alkyl having 1 to 6 carbon atoms, and even more preferably 1 to 4 carbon atoms. The alkyl group may be substituted or unsubstituted. When substituted, the substituent group(s) is preferably one or more individually selected from cycloalkyl, aryl, heteroaryl, heteroalicyclic, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, C-carboxy, O-carboxy, cyanato, isocyanato, thiocyanato, isothiocyanato, nitro, silyl, and amino.

As used herein, the term “halo” refers to chloro, fluoro, bromo, and iodo.

As used herein, the term “hydro” refers to a hydrogen atom (—H group).

As used herein, the term “hydroxy” refers to an —OH group.

As used herein, the term “alkoxy” refers to both an —O-alkyl and an —O-cycloalkyl group, as defined herein. Lower alkoxy refers to —O-lower alkyl groups.

As used herein, the term “aryloxy” refers to both an —O-aryl and an —O-heteroaryl group, as defined herein.

As used herein, the term “mercapto” group refers to an —SH group.

As used herein, the term “alkylthio” group refers to both an S-alkyl and an —S-cycloalkyl group, as defined herein.

As used herein, the term “arylthio” group refers to both an —S-aryl and an —S-heteroaryl group, as defined herein.

As used herein, the term “carbonyl” group refers to a —C(═O)R″ group, where R″ is selected from the group consisting of hydro, alkyl, cycloalkyl, aryl, heteroaryl (bonded through a ring carbon) and heterocyclic (bonded through a ring carbon), as defined herein.

As used herein, the term “aldehyde” group refers to a carbonyl group where R″ is hydro.

As used herein, the term “cycloketone” refer to a cycloalkyl group in which one of the carbon atoms which form the ring has a “═O” bonded to it; i.e. one of the ring carbon atoms is a —C(═O)-group.

As used herein, the term “thiocarbonyl” group refers to a —C(═S)R″ group, with R″ as defined herein.

As used herein, the term “O-carboxy” group refers to a R″C(═O)O-group, with R″ as defined herein.

As used herein, the term “C-carboxy” group refers to a —C(═O)OR″ groups with R″ as defined herein.

As used herein, the term “ester” is a C-carboxy group, as defined herein, wherein R″ is any of the listed groups other than hydro (e.g., methyl, ethyl, lower alkyl).

As used herein, the term “C-carboxy salt” refers to a —C(═O)O⁻M⁺ group wherein M⁺ is selected from the group consisting of lithium, sodium, magnesium, calcium, potassium, barium, iron, zinc and quaternary ammonium.

As used herein, the term “acetyl” group refers to a —C(═O)CH₃ group.

As used herein, the term “carboxyalkyl” refers to —(CH₂)ᵣC(═O)OR″ wherein r is 1-6 and R″ is as defined above.

As used herein, the term “carboxyalkyl salt” refers to a —(CH₂)ᵣC(═O)O⁻M⁺ wherein M⁺ is selected from the group consisting of lithium, sodium, potassium, calcium, magnesium, barium, iron, zinc and quaternary ammonium.

As used herein, the term “carboxylic acid” refers to a C-carboxy group in which R″ is hydro.

As used herein, the term “haloalkyl” refers to an alkyl group substituted with 1 to 6 halo groups, preferably haloalkyl is a —CX₃ group wherein X is a halo group. The halo groups can be independently selected.

As used herein, the term “trihalomethanesulfonyl” refers to a X₃ CS(═O)₂— group with X as defined above.

As used herein, the term “cyano” refers to a —C—N group.

As used herein, the term “cyanato” refers to a —CNO group.

As used herein, the term “isocyanato” refers to a —NCO group.

As used herein, the term “thiocyanato” refers to a —CNS group.

As used herein, the term “isothiocyanato” refers to a —NCS group.

As used herein, the term “sulfinyl” refers to a —S(═O)R″ group, with R″ as defined herein.

As used herein, the term “sulfonyl” refers to a —S(═O)₂ R″ group, with R″ as defined herein.

As used herein, the term “sulfonamido” refers to a —S(═O)₂ NR¹⁷R¹⁸, with R¹⁷ and R¹⁸ as defined herein.

As used herein, the term “trihalomethanesulfonamido” refers to a X₃CS(═O)₂ NR¹⁷-group with X and R¹⁷ as defined herein.

As used herein, the term “O-carbamyl” refers to a —OC(═O)NR¹⁷R¹⁸ group with R¹⁷ and R¹⁸ as defined herein.

As used herein, the term “N-carbamyl” refers to a R¹⁸OC(═O)NR¹⁷-group, with R¹⁷ and R¹⁸ as defined herein.

As used herein, the term “O-thiocarbamyl” refers to a —OC(═S)NR¹⁷R¹⁸ group with R¹⁷ and R¹⁸ as defined herein.

As used herein, the term “N-thiocarbamyl” refers to a R¹⁷OC(═S)NR¹⁸-group, with R¹⁷ and R¹⁸ as defined herein.

As used herein, the term “amino” refers to an —NR¹⁷R¹⁸ group, with R¹⁷ and R¹⁸ both being hydro.

As used herein, the term “C-amido” refers to a —C(═O)NR¹⁷R¹⁸ group with R¹⁷ and R¹⁸ as defined herein. An “N-amido” refers to a R¹⁷C(═O)NR¹⁸— group with R¹⁷ and R¹⁸ as defined herein.

As used herein, the term “nitro” refers to a —NO₂ group.

As used herein, the term “quaternary ammonium” refers to a —⁺NR¹⁷R¹⁸R¹⁹ group wherein R¹⁷, R¹⁸, and R¹⁹ are independently selected from the group consisting of hydro and unsubstituted lower alkyl.

As used herein, the term “methylenedioxy” refers to a —OCH₂O— group wherein the oxygen atoms are bonded to adjacent ring carbon atoms.

As used herein, the term “ethylenedioxy” refers to a —OCH₂CH₂O-group wherein the oxygen atoms are bonded to adjacent ring carbon atoms.

As used herein, “Heterocycle” refers to a mono or bicyclic ring that contains 4-12 atoms, at least one of which is selected from nitrogen, sulfur or oxygen, wherein a —CH₂— group can optionally be replaced by a —C(═O)—, and a ring sulfur atom may be optionally oxidized to form S-oxide(s). Examples of “heterocycles” or “heterocyclic” rings include, but are not limited to, morpholino, piperidyl, piperazinyl, pyrrolidinyl, thiomorpholino, homopiperazinyl, imidazolyl, imidazolidinyl, pyrazolidinyl, dioxanyl and dioxolanyl. “Heterocycle” can include heteroaryls when the pi-electron system of a heterocycle is completely conjugated.

As used herein, “Heteroaryl” refers to a monocyclic or fused ring (i.e., rings which share an adjacent pair of atoms) group having in the ring(s) one or more atoms selected from the group consisting of nitrogen, oxygen and sulfur and, in addition, having a completely conjugated pi-electron system. Examples, without limitation, of heteroaryl groups are pyrrole, furan, thiophene, imidazole, oxazole, thiazole, pyrazole, pyridine, pyrimidine, quinoline, isoquinoline, quinazoline, purine and carbazole.

As used herein, “Aryl” refers to all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl.

As used herein, “Cycloalkyl” refers to an all-carbon monocyclic or fused ring (i.e., rings which share an adjacent pair of carbon atoms) group wherein one or more of the rings does not have a completely conjugated pi-electron system. Examples, without limitation, of cycloalkyl groups are cyclopropane, cyclobutane, cyclopentane, cyclopentene, cyclohexane, adamantane, cyclohexadiene, cycloheptane, and cycloheptatriene.

Methods for Treating Viral Infections

The present invention provides methods for treating viral infection by administering to a patient (either a human or other animal) that is a carrier of a virus a pharmaceutical composition or medicament having a therapeutically effective amount of a compound of Formulae I-III. For example, a carrier of a virus can be identified by conventional diagnostic techniques known in the art, as described above. The identified carrier can be administered with a compound of Formulae I-III, preferably in a pharmaceutical composition having a pharmaceutically acceptable carrier.

In another aspect, the present invention provides methods for treating an active viral infection by administering to a patient (either a human or other animal) that exhibits characteristic symptoms of a viral infection a pharmaceutical composition or medicament having a therapeutically effective amount of a compound of Formulae I-III. Alternatively, the presence of viral infection may be detected or determined directly by any appropriate method in the art. The infected individual so identified can be administered with a compound of Formulae I-III, preferably in a pharmaceutical composition having a pharmaceutically acceptable carrier.

Consequently, the methods of the present invention may be generally useful in treating or preventing diseases or disorders associated with viral infection in animals, particularly humans. Such viral infection can be caused by viruses including, but not limited to, lentiviruses such as human immunodeficiency virus types 1 and 2 (HIV), human T-cell lymphotropic virus type 1 and 2 (HTLV-I and HTLV-II), SIV, EIAV (equine infectious anemia virus), BIV, FIV, CAEV, VMV, and MMLV (Moloney murine leukemia virus). Such viral infections can also be caused by hepatitis A virus, hepatitis B virus, hepatitis C virus, hepatitis D virus, hepatitis E virus, hepatitis G virus, human foamy virus, or by human herpes viruses (e.g., herpes simplex virus type-1, herpes simplex virus type-2, herpes simplex virus type-3 (also known as Varicella-zoster virus), herpes simplex virus type-4 (also known as Epstein Barr virus or EBV), herpes simplex virus type-5, herpes simplex virus type-7). Such viral infections can also be caused by influenza viruses (types A, B or C), human parainfluenza viruses, respiratory syncytial virus, smallpox virus (variola virus), monkeypox virus, vaccinia virus, human papilloma virus, human parechovirus 2, mumps virus, Measles virus, Rubella virus, Semliki Forest virus, West Nile virus, Colorado tick fever virus, foot-and-mouth disease virus, Ebola virus, Marburg virus, polyomavirus, TT virus, Lassa virus, lymphocytic choriomeningitis virus, vesicular stomatitis virus, rotavirus, varicella virus, parvovirus, cytomegalovirus, encephalitis viruses, adenovirus, echovirus, rhinoviruses, filoviruses, coxachievirus, coronavirus (such as SARS-associated coronavirus), Dengue viruses, yellow fever virus, hantaviruses, regional hemorrhagic fever viruses, molluscum virus, polio virus, rabies virus, etc. In some embodiments, the methods are used in treating or preventing infections by enveloped viruses. In specific embodiments, as described below, particular viruses known to infect humans and cause disease are treated by the methods of the present invention.

HIV:

As used herein, the term “HIV infections” generally encompasses infection of a host animal, particularly a human host, by the human immunodeficiency virus (HIV) family of retroviruses including, but not limited to, HIV I (also known as HTLV-III), HIV II (also known as LAV-1), HIV III (also known as LAV-2), and the like. “HIV” can be used herein to refer to any strains, forms, subtypes, clades and variations in the HIV family. Thus, treating HIV infection will encompass the treatment of a person who is a carrier of any of the HIV family of retroviruses or a person who is diagnosed of active AIDS, as well as the treatment or prophylaxis of the AIDS-related conditions in such persons. A carrier of HIV may be identified by any methods known in the art. For example, a person can be identified as HIV carrier on the basis that the person is anti-HIV antibody positive, or is HIV-positive, or has symptoms of AIDS. That is, “treating HIV infection” should be understood as treating a patient who is at any one of the several stages of HIV infection progression, which, for example, include acute primary infection syndrome (which can be asymptomatic or associated with an influenza-like illness with fevers, malaise, diarrhea and neurologic symptoms such as headache), asymptomatic infection (which is the long latent period with a gradual decline in the number of circulating CD4 T-cells), and AIDS (which is defined by more serious AIDS-defining illnesses and/or a decline in the circulating CD4 T-cell count to below a level that is compatible with effective immune function).

As used herein, the term “delaying the onset of HIV infection” means treating an individual who (1) is at risk of infection by HIV, or (2) is suspected of infection by HIV or of exposure to HIV, or (3) has suspected past exposure to HIV, to delay the onset of acute primary infection syndrome by at least three months. As is known in the art, clinical findings typically associated with acute primary infection syndrome may include an influenza-like illness with fevers, malaise, nausea/vomiting/diarrhea, pharyngitis, lymphadenopathy, myalgias, and neurologic symptoms such as headache, encephalitis, etc. The individuals at risk may be people who perform any of following acts: contact with HIV-contaminated blood, blood transfusion, exchange of body fluids, “unsafe” sex with an infected person, accidental needle stick, injection of drug with contaminated needles or syringes, receiving a tattoo or acupuncture with contaminated instruments, or transmission of the virus from a mother to a baby during pregnancy, delivery or shortly thereafter. The term “delaying the onset of HIV infection” may also encompass treating a person who has not been diagnosed as having HIV infection but is believed to be at risk of infection by HIV, or has been exposed to HIV through contaminated blood, etc.

In addition, the term “delay the onset of AIDS” means delaying the onset of AIDS (which is characterized by more serious AIDS-defining illnesses and/or a decline in the circulating CD4 cell count to below a level that is compatible with effective immune function, i.e. below about 200 μl) and/or AIDS-related conditions, by treating an individual (1) at risk of infection by HIV, or suspected of being infected with HIV, or (2) having HIV infection but not AIDS, to delay the onset of AIDS by at least six months. Individuals at risk of HIV infection may be those who are suspected of past exposure, or considered to be at risk of present or future exposure, to HIV by, e.g., contact with HIV-contaminated blood, blood transfusion, transplantation, exchange of body fluids, “unsafe” sex with an infected person, accidental needle stick, receiving a tattoo or acupuncture with contaminated instruments, or transmission of the virus from a mother to a baby during pregnancy, delivery or shortly thereafter.

The term “treating AIDS” means treating a patient who exhibits more serious AIDS-defining illnesses and/or a decline in the circulating CD4 cell count to below a level that is compatible with effective immune function (typically below about 200/μl). The term “treating AIDS” also encompasses treating AIDS-related conditions, which means disorders and diseases incidental to or associated with AIDS or HIV infection such as AIDS-related complex (ARC), progressive generalized lymphadenopathy (PGL), anti-HIV antibody positive conditions, and HIV-positive conditions, AIDS-related neurological conditions (such as dementia or tropical paraparesis), Kaposi's sarcoma, thrombocytopenia purpurea and associated opportunistic infections such as Pneumocystis carinii pneumonia, Mycobacterial tuberculosis, esophageal candidiasis, toxoplasmosis of the brain, CMV retinitis, HIV-related encephalopathy, HIV-related wasting syndrome, etc.

HBV:

As used herein, the term “HBV infection” generally encompasses infection of a human by any strain or serotype of hepatitis B virus, including acute hepatitis B infection and chronic hepatitis B infection. Thus, treating HBV infection means the treatment of a person who is a carrier of any strain or serotype of hepatitis B virus, or a person who is diagnosed with active hepatitis B, to reduce the HBV viral load in that person or to alleviate one or more symptoms associated with HBV infection and/or hepatitis B, including, e.g., nausea and vomiting, loss of appetite, fatigue, muscle and joint aches, elevated transaminase blood levels, increased prothrombin time, jaundice (yellow discoloration of the eyes and body) and dark urine. A carrier of HBV may be identified by any method known in the art. For example, a person can be identified as HBV carrier on the basis that the person is anti-HBV antibody positive (e.g., based on hepatitis B core antibody or hepatitis B surface antibody), or is HBV-positive (e.g., based on hepatitis B surface antigens (HBeAg or HbsAg) or HBV RNA or DNA) or has symptoms of hepatitis B infection or hepatitis B. Hence, “treating HBV infection” should be understood as treating a patient who is at any one of the several stages of HBV infection progression. In addition, the term “treating HBV infection” will also encompass treating individuals with a suspected HBV infection after suspected exposure to HBV by, e.g., contact with HBV-contaminated blood, blood transfusion, exchange of body fluids, “unsafe” sex with an infected person, accidental needle stick, receiving a tattoo or acupuncture with contaminated instruments, or transmission of the virus from a mother to a baby during pregnancy, delivery or shortly thereafter. The term “treating HBV infection” will also encompass treating a person who is free of HBV infection but is believed to be at risk of infection by HBV.

In yet another aspect, a method of treating HBV infection in a patient co-infected with HBV and HIV is provided by administering a therapeutically effective amount of a compound according to Formulae I-III to such a patient. Particularly, HIV infection is associated with an approximate threefold increase in the development of persistent hepatitis B. The compounds according to Formulae I-III are particularly suitable for patients co-infected with HIV and HBV. The presently marketed drug interferon alpha is not effective in treating HBV and HIV co-infection. Lamivudine and some other reverse transcriptase inhibitors are useful in treating such co-infections, but Lamivudine is particularly toxic and can cause hepatic injury which worsens hepatitis B. In addition, such reverse transcriptase inhibitors often must be used in cocktails. In contrast, the compounds according to the present invention are significantly less toxic, and are less likely to result in evolved viral resistance. Thus, in accordance with the present invention, a compound according to Formulae I-III is administered alone, or in combination with another anti-HIV or anti-HBV drug, in a therapeutically effective amount to a mammal, particularly a human co-infected with both HBV and HIV. The method may include a step of identifying a patient co-infected with HBV and HIV by techniques commonly known in the art. For example, PCR tests can be used to detect HBV DNA or RNA and HIV RNA in blood samples obtained from a test subject. Alternatively, virus-specific antibodies or antigens may be also employed for the detection of HBV and HIV infection.

The term “preventing hepatitis B” as used herein means preventing in a patient who has an HBV infection, is suspected to have an HBV infection, or is at risk of contracting an HBV infection, from developing hepatitis B (which are characterized by more serious hepatitis-defining symptoms), cirrhosis, or hepatocellular carcinoma.

HCV:

As used herein, the term “HCV infection” generally encompasses infection of a human by any types or subtypes of hepatitis C virus, including acute hepatitis C infection and chronic hepatitis C infection. Thus, treating HCV infection means the treatment of a person who is a carrier of any types or subtypes of hepatitis C virus, or a person who is diagnosed with active hepatitis C, to reduce the HCV viral load in that person or to alleviate one or more symptoms associated with HCV infection and/or hepatitis C. A carrier of HCV may be identified by any methods known in the art. For example, a person can be identified as HCV carrier on the basis that the person is anti-HCV antibody positive, or is HCV-positive (e.g., based on HCV RNA or DNA) or has symptoms of hepatitis C infection or hepatitis C (e.g., elevated serum transaminases). Hence, “treating HCV infection” should be understood as treating a patient who is at any one of the several stages of HCV infection progression. In addition, the term “treating HCV infection” will also encompass treating individuals with a suspected HCV infection after suspected past exposure to HCV by, e.g., contact with HCV-contaminated blood, blood transfusion, exchange of body fluids, “unsafe” sex with an infected person, accidental needle stick, receiving a tattoo or acupuncture with contaminated instruments, or transmission of the virus from a mother to a baby during pregnancy, delivery or shortly thereafter. The term “treating HCV infection” will also encompass treating a person who is free of HCV infection but is believed to be at risk of infection by HCV. The term of “preventing HCV” as used herein means preventing in a patient who has HCV infection or is suspected to have HCV infection or is at risk of HCV infection from developing hepatitis C (which is characterized by more serious hepatitis-defining symptoms), cirrhosis, or hepatocellular carcinoma.

Importantly, about one quarter of all HIV-infected persons in the United States, or an estimated 200,000 people, are infected with both HCV and HIV (See National Center for HIV, STD and TB Prevention report at http://www.cdc.gov/hiv/pubs/facts/HIV-HCV_Coinfection.htm and Thomas, D. L. Hepatology 36:S201-S209 (2002)). As the lives of HIV-infected persons have been prolonged by use of highly active antiretroviral therapy, liver disease has emerged as an important, and in some settings, the leading cause of morbidity and mortality. HIV infection appears to adversely affect all stages of HCV infection. Particularly, HIV infection is associated with a significant increase in the development of persistent hepatitis C, with higher titers of HCV, more rapid progression to HCV-related liver disease, and an increased risk for HCV-related cirrhosis (scarring) of the liver. In turn, HCV may affect the management of HIV infection, increasing the incidence of liver toxicity caused by antiretroviral medications (Thomas, D. L. Hepatology 36:S201-S209, (2002) and National Center for HIV, STD and TB Prevention report at http://www.cdc.gov/hiv/pubs/facts/HIV-HCV_Coinfection.htm).

In the United States, two different treatment regimens have been approved as therapy for chronic hepatitis C: monotherapy with alpha interferon and combination therapy with alpha interferon and ribavirin. Among HIV-negative persons with chronic hepatitis C, combination therapy consistently yields higher rates (30%-40%) of sustained response than monotherapy (10%-20%). Combination therapy is more effective against viral genotypes 2 and 3, and requires a shorter course of treatment; however, viral genotype 1 is the most common among U.S. patients. Combination therapy is associated with more side effects than monotherapy, but, in most situations, it is preferable. At present, interferon monotherapy is reserved for patients who have contraindications to the use of ribavirin. (See, http://www.cdc.gov/hiv/pubs/facts/HIV-HCV_Coinfection.htm)

Hence, in yet another aspect, a method of treating HCV infection in a patient co-infected with HCV and HIV is provided by administering a therapeutically effective amount of a compound according to Formulae I-III to such a patient. The compounds according to Formulae I-III are particularly suitable for patients co-infected with HIV and HCV. Particularly, the compounds are especially effective in inhibiting HCV infection and/or egress from host cells. Moreover, the compounds can also be effective in inhibiting HIV entry into and/or egress from host cells. In contrast to the combination therapy described above, the compounds according to the present invention can be significantly less toxic, and less likely to result in evolved viral resistance. Thus, in accordance with the present invention, a compound according to Formulae I-III is administered alone, or in combination with another anti-HIV or anti-HCV drug, in a therapeutically effective amount to a mammal, particularly a human co-infected with both HCV and HIV. The method may include a step of identifying a patient co-infected with HCV and HIV by techniques commonly known in the art. For example, PCR tests can be used to detect HCV DNA or RNA and HIV RNA in blood samples obtained from a test subject. Alternatively, virus-specific antibodies or antigens may be also employed for the detection of HCV and HIV infection.

Herpes Viruses:

Herpes viruses are one of the most common human pathogens. Members of the herpesvirus family include herpes simplex virus type-1 (HSV-1), herpes simplex virus type-2 (HSV-2), Varicella-zoster virus (herpes simplex virus type-3 or HSV-3; also known as chicken pox), and Epstein-Barr virus (herpes simplex virus type-4 or HSV-4). HSV-1 commonly causes herpes labialis (also called oral herpes, cold sores, fever blisters), which are highly infectious open sores that crust over before healing. HSV-1 can also cause eye and brain infection. HSV-2 commonly causes genital herpes. HSV-1 can also cause genital herpes, though far less frequently than HSV-2. After an initial infectious cycle, HSV-1 and HSV-2 generally establish life-long latent infections in sensory neurons near the site of infection. These latent infections exist without showing any signs or symptoms of infection or disease, until some event reactivates the virus. Reactivation generally causes recurrent lesions close to, or in the same location as, the site of initial infection. Reactivation seems to occur during periods of emotional stress, or periods of reduced immune system function.

In addition to oral and genital herpes, HSV-1 and HSV-2 can cause other diseases. Examples of such diseases include herpes simplex encephalitis—a rare but potentially fatal herpetic infection of the brain; neonatal herpes,—a rare but potentially severe HSV infection in newborns (resulting from transmission of the virus from the mother to the baby during delivery); herpetic whitlow—an HSV infection of the finger (acquired either from transfer of the infection from another part of the body or from direct contact with another party having an HSV infection); and herpes keratitis—an HSV infection of the eye (one of the most common causes of blindness). Thus, herpes simplex virus infection of humans is a significant health problem.

Genital herpes is primarily treated with suppressive and episodic therapies. Suppressive therapy is used to treat outbreaks before they occur, while episodic therapy treats outbreaks when they occur. Treatment with valacyclovir HCl, acyclovir, and famciclovir, can be used in both suppressive and episodic therapies.

Currently there is no known cure for HSV-1 infection. The available antiviral therapies are not completely effective and there is a chance that the virus will become resistant to the treatment. Thus, there is a clear need for improved methods and compositions for treating HSV-1.

Epstein-Barr virus (herpes simplex virus-4), hereafter referred to as “EBV”, occurs worldwide. In fact, most people become infected with EBV during their lives. A large percentage of adults in the United States have been infected. Infants are susceptible to EBV as soon as maternal antibody protection present at birth disappears. Many children become infected with EBV, and these infections usually cause no symptoms. The symptoms of EBV infection in children can be indistinguishable from the symptoms of other typical childhood illnesses. Individuals not infected as a child have a risk of being infected during adolescence or young adulthood, which often causes infectious mononucleosis (mono). Symptoms of infectious mononucleosis include fever, sore throat, and swollen lymph glands, less often a swollen spleen or liver involvement may develop. Rarely, heart problems or involvement of the central nervous system occur. Infectious mononucleosis is almost never fatal. The symptoms of infectious mononucleosis usually resolve in 1 or 2 months, but EBV remains dormant or latent in a few cells in the throat and blood for the rest of the infected person's life. Periodically, the virus can reactivate and is commonly found in the saliva of infected persons. Reactivation usually occurs without symptoms of illness.

EBV is thought to be associated with a number of other diseases including Burkitt's lymphoma, nasopharyngeal carcinoma, and Hodgkin's disease. Diseases caused by EBV are particularly common among people with reduced immunity. EBV is associated with a tumor often found in organ transplant patients that is referred to as post-transplant lymphoproliferative disease. The immune systems of such patients are usually artificially suppressed by drug therapy to help prevent the body from rejecting the new organ. Individuals infected with HIV, and have AIDS, also have reduced immunity and commonly suffer from oral hairy leukoplakia, a condition involving considerable replication of EBV in cells along the edge of the tongue. It has also been suggested that the high incidence of malaria in countries where Burkitt's lymphoma is prevalent may also play a role in the disease by suppressing the body's immune system.

Scientists are finding it difficult to explain why the virus causes a relatively mild disease like glandular fever in some people and malignant tumors in others. Genetic factors may play a role. Regardless, treatments are needed to combat EBV.

As used herein, the terms “herpes simplex virus” or HSV refers to any strain of herpes simplex virus, including, but not limited to HSV-1, HSV-2, HSV-3 (Varicella-zoster virus or chicken pox), and HSV-4 (or EBV). Thus, “treating HSV infections” will encompass the treatment of a person who is actively infected with, or carrier of a latent infection of, any of the HSV family of herpes viruses.

As used herein, the term “HSV infection” generally encompasses infection of a human by any strain of herpes simplex virus, and includes both active and latent infections. Thus, “treating HSV infection” means the treatment of a person who is a carrier of any strain of HSV. For example, a person can be identified as an HSV carrier on the basis that the person is anti-HSV antibody positive or has symptoms of an HSV infection. Hence, “treating HSV infection” should be understood as treating a patient who is at any one of the several stages of HSV infection progression. In addition, the term “treating HSV infection” will also encompass treating individuals with a suspected HSV infection after suspected exposure to HSV by, e.g., contact with HSV-contaminated blood, blood transfusion, exchange of body fluids, “unsafe” sex with an infected person, accidental needle stick, receiving a tattoo or acupuncture with contaminated instruments, or transmission of the virus from a mother to a baby during pregnancy, delivery or shortly thereafter. The term “treating HSV infection” will also encompass treating a person who is free of HSV infection but is believed to be at risk of infection by HSV.

In yet another aspect, a method of treating HSV infection in a patient co-infected with HSV and HIV is provided by administering a therapeutically effective amount of a compound according to Formulae I-III to such a patient. Particularly, HIV infection is associated with an increase in active HSV infections, presumably due to the immunocompromised state created by the HIV infection. The compounds according to Formulae I-III are particularly suitable for patients co-infected with HIV and HSV. The presently marketed drug interferon alpha is not effective in treating HBV and HIV co-infection. Lamivudine and some other reverse transcriptase inhibitors are useful in treating such co-infections, but Lamivudine is particularly toxic and can cause hepatic injury which worsens hepatitis B. In addition, such reverse transcriptase inhibitors often must be used in cocktails. In contrast, the compounds according to the present invention are significantly less toxic, and are less likely to result in evolved viral resistance. Thus, in accordance with the present invention, a compound according to Formulae I-III is administered alone, or in combination with another anti-HIV or anti-HSV drug, in a therapeutically effective amount to a mammal, particularly a human co-infected with both HSV and HIV. The method may include a step of identifying a patient co-infected with HSV and HIV by techniques commonly known in the art. For example, PCR tests can be used to detect HSV DNA or RNA and HIV RNA in blood samples obtained from a test subject. Alternatively, virus-specific antibodies or antigens may be also employed for the detection of HSV and HIV infection.

The term “delaying the onset of HSV-associated symptoms” as used herein means preventing in a patient who has an HSV infection, is suspected to have an HSV infection, or is at risk of contracting an HSV infection, from developing oral herpes, genital herpes, chickenpox or shingles, or a chronic EBV infection.

Influenza:

Influenza infection is associated with an average of 36,000 deaths and 114,000 hospitalizations per year in the United States alone. Although there are three recognized types of influenza viruses, influenza A, B, and C, types A and B are responsible for annual winter flu epidemics. Influenza A infects many different animal species besides humans, including ducks, chickens, pigs, whales, horses, and seals. Influenza B viruses generally only infect humans.

All three types of influenza virus have genomes composed of eight different RNA helices, which encodes a single gene and are bound by a nucleoprotein that determines the viral type: A, B, or C. In effect, the influenza genome is made up of eight separate pieces of nucleic acid that can come together to form viruses with new combinations of viral genes when cells become co-infected by more than one viral type. Two of these RNA helices encode the important viral surface proteins hemagglutinin and neuramidase, which are embedded in the lipid bilayer of a mature virus particle.

Variations in the viral hemagglutinin and neuramidase determine the viral subtype. Hemagglutinin is responsible for entry of the virus into the host cell, while neuramidase is important in the release of newly formed viruses from the infected cells. Antibodies to hemagglutinin can neutralize the virus and are the major determinant for immunity. Antibodies to neuramidase do not neutralize the virus but may limit viral replication and the course of infection. Host antibodies to specific types of hemagglutinin and neuramidase prevent and generally ameliorate future infection by the same viral strain. However, since the genetic makeup of viral strains is dynamic and ever-changing, immunity gained through successful resistance to one strain gained during an infection one year may be useless in combating a new, recombined, variant strain the next year.

Epidemics of influenza are thought to result when viral strains change over time by the process of antigenic drift. Antigenic drift (caused by mutations in the principal viral antigen genes, especially in the hemagglutinin or neuramidase genes) results in small changes in surface antigens, and occurs essentially continuously over time. When these changes occur in the right places in the genes, they render the new antigens unrecognizable by the antibodies raised against other influenza virus strains during previous infections.

Influenza pandemics (or worldwide epidemics) occur as a result of “antigenic shift.” Antigenic shift is an abrupt, major change in an influenza A virus that results from a new hemagglutinin and/or new hemagglutinin and neuraminidase protein appearing in an influenza A strain. Such shifts are generally thought to occur when a new combination of viral genomic RNAs is created, possibly in a non-human species, and that new combination is passed to humans. When such an antigenic shift occurs, most humans have little or no protection against the virus, and an infection can prove lethal.

Influenza pandemics have resulted in massive loss of life during the history of man. The influenza pandemic of 1918-1919 resulted in the deaths of about 20-40 million people. In support of the antigenic shift hypothesis presented above, molecular analyses recently demonstrated that the influenza virus responsible for the 1918-19 pandemic is related to a swine influenza virus that belongs to the same family of influenza virus that still causes the flu in humans today.

Two categories of treatment/preventative strategies are available for influenza infection: vaccination with “the flu shot” and administration of antiviral drugs. The flu shot involves vaccination with killed or inactivated influenza viruses. The antiviral drugs available for treating influenza infection including amantadine, rimantadine, zanamivir, and osteltamivir. Amantadine and rimantadine are used for treating and preventing influenza A infection, zanamivir is used for treating influenza A and B infection, and osteltamivir is used for treating and preventing influenza A and B infection.

Despite the numerous drugs and vaccinations available, there is a need for improved methods and compositions for both treating and preventing influenza infection.

As used herein, the term “influenza” and “influenza virus” refer to any type or subtype of influenza, including types A, B and C, and all subtypes thereof. Consequently, the term “influenza infection” encompasses infection by any strain of influenza, and the term “treating influenza infection” is understood to mean the treatment of an animal, particularly a human, infected by any strain of influenza. In addition, the term “treating influenza infection” will also encompass treating individuals with a suspected influenza infection after suspected exposure to influenza. The term “treating influenza infection” will also encompass treating a person who is apparently free of an influenza infection but is believed to be at risk of infection by influenza.

Poxviruses:

As used herein, the terms “smallpox virus” or “variola virus” refers to any strain of smallpox virus including variola major and variola minor (also referred to as alastrim). Examples of such human variola virus isolates are well known and the complete genomic nucleotide sequence one strain has been determined (See, e.g., Harrison's 15th Edition Principles of Internal Medicine, Braunwald et al. EDS. McGraw-Hill, United States, and Genbank accession no. NC_{—}001611). Skilled artisans are capable of diagnosing individuals infected or suspected of being infected with smallpox. The term “treating smallpox” or “treating variola virus” refers to both treating the symptoms of the disease as well as reducing the viral load, infectivity and/or replication of the virus. The term of “delaying the onset of symptoms associated with smallpox infection” as used herein means treating a patient who is free of smallpox infection, or is believed to be at risk of infection by smallpox, or is infected with smallpox to delay the onset of one or more symptoms associated with smallpox infection by at least 3 months. The term “treating smallpox” also encompasses treating a person who either has smallpox infection, is suspected to have smallpox infection, or is at risk of developing smallpox from a smallpox virus infection (which is characterized by more serious smallpox-defining symptoms like macular rash, fever, vesicular lesions and pustular lesions).

An outbreak of monkeypox occurred for the first time in the United States in June of 2003. The causative agent is the monkeypox virus, which belongs to the group of viruses that includes the smallpox virus (variola), the virus used in the smallpox vaccine (vaccinia), and the cowpox virus. In humans, the signs and symptoms of monkeypox are like those of smallpox, but usually much milder, although monkeypox, unlike smallpox causes the lymph nodes to swell. In Africa, where most cases of monkeypox are known to occur, infections result in deaths of between 1% and 10% of infected individuals. As used herein, the term “treating monkeypox” or “treating monkeypox virus” refers to both treating the symptoms of the disease as well as reducing the viral load, infectivity and/or replication of the virus. The term of “preventing monkeypox infection” as used herein means preventing infection in a patient who is free of monkeypox infection but is believed to be at risk of infection by monkeypox. The term of “delaying the onset of symptoms associated with monkeypox infection” as used herein means treating a patient who is free of monkeypox infection, or is believed to be at risk of infection by monkeypox, or is infected with monkeypox to delay the onset of one or more symptoms associated with monkeypox infection by at least 3 months.

Coronaviruses:

As used herein, the terms “SARS-CoV”, “SARS” or “SARS-associated Coronavirus” refers to any strain of coronavirus associated with severe acute respiratory syndrome. Examples of such human coronavirus isolates are known as HCoV-OC43 and HCoV-229E (See, e.g., Marra et al. Science 300:1399 (2003) and Rota et al. Science 300:1394 (2003)(Genbank accession no. AY278741). Skilled artisans are capable of diagnosing individuals infected or suspected of being infected with a SARS associated Coronavirus. The term “treating SARS” or “treating SARS associated Cornoavirus” refers to both treating the symptoms of the disease, as well as reducing the infectivity and/or replication of the SARS-associated Coronavirus. The term “treating SARS” also encompasses treating a person who is free of SARS-CoV infection but is believed to be at risk of infection by SARS-CoV. The term of “preventing SARS” as used herein means preventing in a patient who has SARS-CoV infection or is suspected to have SARS-CoV infection or is at risk of SARS-CoV infection from developing SARS (which is characterized by more serious SARS-CoV-defining symptoms like severe respiratory illness, fever, dry nonproductive cough, shortness of breath, and atypical pneumonia).

West Nile virus:

West Nile (WN) virus has emerged in recent years in temperate regions of Europe and North America, presenting a threat to public, equine, and animal health. The most serious manifestation of WN virus infection is fatal encephalitis (inflammation of the brain) in humans and horses, as well as mortality in certain domestic and wild birds. WN virus infection is a growing problem in North America. During 2002 in the United States alone, there were 4,156 documented cases of WN virus infections of humans and 284 deaths. As used herein, the terms “treating West Nile virus,” “treating West Nile disease” refer to treating the symptoms of the disease in both known and suspected cases of WN virus infection.

In one embodiment, the methods of treatment are generally used to treat an individual experiencing an active viral infection, whether acute or chronic, by any of the aforementioned viruses. In another embodiment, the methods are generally used for treating a carrier of any of the aforementioned viruses who is not experiencing an active viral outbreak. In yet another embodiment, the methods are generally used to treat an individual who is known or suspected to have been exposed to any of the aforementioned viruses. In still another embodiment, the methods are generally used to prophylactically treat an individual who is likely to be exposed to, or is at risk of being exposed to, any of the aforementioned viruses, and thereby prevent infection or lessen its symptoms.

In one particular embodiment, the methods are used for treating an HIV carrier who is not diagnosed as having developed AIDS (which is characterized by more serious AIDS-defining illnesses and/or a decline in the circulating CD4 cell count to below a level that is compatible with effective immune function, i.e., below about 200/μl). For example, the methods can be used in treating a patient at any stages the HIV infection prior to diagnosis of AIDS, including acute HIV syndrome (or acute primary HIV infection syndrome) and asymptomatic infection (which is the long latent period with a gradual decline in the number of circulating CD4 T cells).

In one aspect, the present invention provides methods for treating viral infection—at any stage, and caused by any of the aforementioned viruses, and particularly HIV—in patients who have been, or are being, treated with one or more established antiviral drugs. Examples of such other antiviral compounds include, but are not limited to, protease inhibitors, nucleoside reverse transcriptase inhibitors, non-nucleoside reverse transcriptase inhibitors, integrase inhibitors, fusion inhibitors, and combinations thereof. The compounds of Formulae I-III can be administered to patients who do not respond well to other antiviral drugs (e.g., non-responding, or developing viral resistance) or who experience relapses after treatment with one or more other antiviral drugs or regimens. As used herein, “non-responding patient” or patient “who does not respond well to other antiviral drugs” connote professional observations or judgment by a physician under relevant medical standard or customary practice in the field of antiviral infection therapy. For example, in the case of HIV, a patient may be characterized as non-responding or not responding well if his or her plasma HIV RNA level (or equivalent thereof) does not substantially decrease after treatment with one or more other anti-HIV drugs for a sufficient period of time, or if the reduction of plasma HIV RNA level (or equivalent thereof) is less than a tenfold drop by 4 weeks following the initiation of therapy. Other indications for non-responding patients may include, e.g., persistent decline of CD4 T-cell numbers, adverse drug reaction or toxicity, and clinical deterioration. Thus, the method of the present invention includes a step of identifying such a patient and subsequently administering to the patient a pharmaceutical composition or medicament having a therapeutically effective amount of a compound of Formulae I-III.

In another embodiment, a compound of Formulae I-III is administered to a patient who has undergone a treatment with one or more drugs that target a viral protein such as viral protease, reverse transcriptase, integrase, envelope protein (e.g., gp120 and gp41 for anti-fusion or homolog thereof), and has not responded well to the treatment. The compounds of the present invention belong to a novel class of antiviral drug that is believed to target certain host cell protein(s). Their mode of action is distinct from other antiviral drugs. Thus, they can be especially effective in treating virus-infected patients who do not respond to one or more other antiviral drugs of a different class or who experience relapse after treatment with one or more antiviral drugs of a different class.

In addition, the present invention further provides methods for delaying the onset of acute infection comprising administering a pharmaceutical composition or medicament having a prophylactically effective amount of a compound of Formulae I-III to an individual having an acute viral infection or at risk of viral infection or at risk of developing symptomatic infection. For example, in delaying the onset of symptomatic infection, an individual infected with a virus or at risk of viral infection can be identified, and administered with a prophylactically effective amount of a compound according to Formulae I-III, that is, an amount sufficient to delay the onset of acute viral infection by at least six months. Preferably, an amount is used sufficient to delay the onset of acute viral infection by at least 12 months, 18 months or 24 months.

In addition, the present invention also provides methods for delaying the onset of a symptomatic viral infection comprising identifying an individual who (1) is at risk of infection by a virus, or (2) is suspected of infection by a virus or of exposure to a virus, or (3) has a suspected past exposure to a virus, and administering to the individual a pharmaceutical composition or medicament having a prophylactically effective amount of a compound of Formulae I-III.

For purposes of preventing viral infection, treating asymptomatic viral infection, delaying the onset of symptomatic viral infection, or treating symptomatic viral infection, a compound of the present invention may be used in combination with one or more other antiviral compounds, preferably other antiviral compounds that act through different mechanisms of action. Examples of such other antiviral compounds include, but are not limited to, protease inhibitors, nucleoside reverse transcriptase inhibitors, non-nucleoside reverse transcriptase inhibitors, integrase inhibitors, fusion inhibitors, and a combination thereof. “Co-administration or co-administering” means that the active pharmaceutical agents are administered together as a part of the same therapeutic or treatment regime. The active pharmaceutical agents can be administered separately at different times of the day or at the same time. Additionally, the present invention also provides a pharmaceutical composition having a compound according to Formula I and a compound selected from protease inhibitors, nucleoside reverse transcriptase inhibitors, non-nucleoside reverse transcriptase inhibitors, integrase inhibitors, fusion inhibitors, immunomodulators, vaccines, and combinations thereof. However, it is to be understood that such other antiviral compounds should not interfere with, or adversely affect, the intended effects of the active compounds of this invention. Co-administering to an individual in need of treatment a therapeutically effective amount of a compound of Formulae I-III and a therapeutically effective amount of one or more other antiviral compounds provide a method according to this aspect of the invention.

Accordingly, the present invention also provides pharmaceutical compositions or medicaments useful for the above treatment and prevention purposes and having a therapeutically effective amount of a compound according to Formula I and a therapeutically effective amount of one or more other antiviral compounds. Preferably, such other antiviral compounds have a different mode of action than that of the compounds according to Formulae I-III. More preferably, such other antiviral compounds target a viral protein. Examples of such compounds include, but are not limited to, protease inhibitors, nucleoside reverse transcriptase inhibitors, non-nucleoside reverse transcriptase inhibitors, integrase inhibitors, fusion inhibitors, and combinations thereof.

The present invention further provides an article of manufacture comprising a pharmaceutical composition or medicament having a therapeutically or prophylactically effective amount of a compound according to Formulae I-III. The pharmaceutical composition or medicament can be in a container such as bottle, gel capsule, vial or syringe. The article of manufacture may also include instructions for the use of the pharmaceutical composition or medicament in the various antiviral applications provided above. The instructions can be printed on paper, or in the form of a pamphlet or book. Preferably, the article of manufacture according to the present invention further comprises a therapeutically or prophylactically effective amount of one or more other antiviral compounds as described above.

Typically, compounds according to Formulae I-III can be effective at an amount of from about 0.01 μg/kg to about 100 mg/kg per day based on total body weight. The active ingredient may be administered at once, or may be divided into a number of smaller doses to be administered at predetermined intervals of time. The suitable dosage unit for each administration can be, e.g., from about 1 μg to about 2000 mg, preferably from about 5 μg to about 1000 mg. In the case of combination therapy, a therapeutically effective amount of one or more other antiviral compounds can be administered in a separate pharmaceutical composition, or alternatively included in the pharmaceutical composition according to the present invention which contains a compound according to Formulae I-III. The pharmacology and toxicology of many of such other antiviral compounds are known in the art. See e.g., Physicians Desk Reference, Medical Economics, Montvale, N.J.; and The Merck Index, Merck & Co., Rahway, N.J. The therapeutically effective amounts and suitable unit dosage ranges of such compounds used in art can be equally applicable in the present invention.

It should be understood that the dosage ranges set forth above are exemplary only and are not intended to limit the scope of this invention. The therapeutically effective amount for each active compound can vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like, as will be apparent to a skilled artisan. The amount of administration can be adjusted as the various factors change over time.

In the pharmaceutical compositions, the active agents can be in any pharmaceutically acceptable salt form. As used herein, the term “pharmaceutically acceptable salts” refers to the relatively non-toxic, organic or inorganic salts of the active compounds, including inorganic or organic acid addition salts of the compound. Examples of salts of basic active ingredient compounds include, but are not limited to, hydrochloride salts, hydrobromide salts, sulfate salts, bisulfate salts, nitrate salts, acetate salts, phosphate salts, nitrate salts, oxalate salts, valerate salts, oleate salts, borate salts, benzoate salts, laurate salts, stearate salts, palmitate salts, lactate salts, tosylate salts, citrate salts, maleate, salts, succinate salts, tartrate salts, napththylate salts, fumarate salts, mesylate salts, laurylsuphonate salts, glucoheptonate salts, and the like. See, e.g., Berge, et al. J. Pharm. Sci., 66:1-19 (1977). Examples of salts of acidic active ingredient compounds include, e.g., alkali metal salts, alkaline earth salts, and ammonium salts. Thus, suitable salts may be salts of aluminum, calcium, lithium, magnesium, potassium, sodium and zinc. In addition, organic salts may also be used including, e.g., salts of lysine, N,N′-dibenzylethylenediamine, chloroprocaine, choline, diethanolamine, ethylenediamine, meglumine (N-methylglucamine), procaine and tris.

For oral delivery, the active compounds can be incorporated into a formulation that includes pharmaceutically acceptable carriers such as binders (e.g., gelatin, cellulose, gum tragacanth), excipients (e.g., starch, lactose), lubricants (e.g., magnesium stearate, silicon dioxide), disintegrating agents (e.g., alginate, Primogel, and corn starch), and sweetening or flavoring agents (e.g., glucose, sucrose, saccharin, methyl salicylate, and peppermint). The formulation can be orally delivered in the form of enclosed gelatin capsules or compressed tablets. Capsules and tablets can be prepared in any conventional techniques. The capsules and tablets can also be coated with various coatings known in the art to modify the flavors, tastes, colors, and shapes of the capsules and tablets. In addition, liquid carriers such as fatty oil can also be included in capsules.

Suitable oral formulations can also be in the form of suspension, syrup, chewing gum, wafer, elixir, and the like. If desired, conventional agents for modifying flavors, tastes, colors, and shapes of the special forms can also be included. In addition, for convenient administration by enteral feeding tube in patients unable to swallow, the active compounds can be dissolved in an acceptable lipophilic vegetable oil vehicle such as olive oil, corn oil and safflower oil.

The active compounds can also be administered parenterally in the form of solution or suspension, or in lyophilized form capable of conversion into a solution or suspension form before use. In such formulations, diluents or pharmaceutically acceptable carriers such as sterile water and physiological saline buffer can be used. Other conventional solvents, pH buffers, stabilizers, anti-bacteria agents, surfactants, and antioxidants can all be included. For example, useful components include sodium chloride, acetates, citrates or phosphates buffers, glycerin, dextrose, fixed oils, methyl parabens, polyethylene glycol, propylene glycol, sodium bisulfate, benzyl alcohol, ascorbic acid, and the like. The parenteral formulations can be stored in any conventional containers such as vials and ampoules.

Routes of topical administration include nasal, bucal, mucosal, rectal, or vaginal applications. For topical administration, the active compounds can be formulated into lotions, creams, ointments, gels, powders, pastes, sprays, suspensions, drops and aerosols. Thus, one or more thickening agents, humectants, and stabilizing agents can be included in the formulations. Examples of such agents include, but are not limited to, polyethylene glycol, sorbitol, xanthan gum, petrolatum, beeswax, or mineral oil, lanolin, squalene, and the like. A special form of topical administration is delivery by a transdermal patch. Methods for preparing transdermal patches are disclosed, e.g., in Brown, et al., Annual Review of Medicine, 39:221-229 (1988), which is incorporated herein by reference.

Subcutaneous implantation for sustained release of the active compounds may also be a suitable route of administration. This entails surgical procedures for implanting an active compound in any suitable formulation into a subcutaneous space, e.g., beneath the anterior abdominal wall. See, e.g., Wilson et al., J. Clin. Psych. 45:242-247 (1984). Hydrogels can be used as a carrier for the sustained release of the active compounds. Hydrogels are generally known in the art. They are typically made by crosslinking high molecular weight biocompatible polymers into a network, which swells in water to form a gel like material. Preferably, hydrogels are biodegradable or biosorbable. For purposes of this invention, hydrogels made of polyethylene glycols, collagen, or poly(glycolic-co-L-lactic acid) may be useful. See, e.g., Phillips et al., J. Pharmaceut. Sci., 73:1718-1720 (1984).

The active compounds can also be conjugated, to a water soluble non-immunogenic non-peptidic high molecular weight polymer to form a polymer conjugate. For example, an active compound is covalently linked to polyethylene glycol to form a conjugate. Typically, such a conjugate exhibits improved solubility, stability, and reduced toxicity and immunogenicity. Thus, when administered to a patient, the active compound in the conjugate can have a longer half-life in the body, and exhibit better efficacy. See generally, Burnham, Am. J. Hosp. Pharm., 15:210-218 (1994). PEGylated proteins are currently being used in protein replacement therapies and for other therapeutic uses. For example, PEGylated interferon (PEG-INTRON A®) is clinically used for treating Hepatitis B. PEGylated adenosine deaminase (ADAGEN®) is being used to treat severe combined immunodeficiency disease (SCIDS). PEGylated L-asparaginase (ONCAPSPAR®) is being used to treat acute lymphoblastic leukemia (ALL). It is preferred that the covalent linkage between the polymer and the active compound and/or the polymer itself is hydrolytically degradable under physiological conditions. Such conjugates known as “prodrugs” can readily release the active compound inside the body. Controlled release of an active compound can also be achieved by incorporating the active ingredient into microcapsules, nanocapsules, or hydrogels generally known in the art.

Liposomes can also be used as carriers for the active compounds of the present invention. Liposomes are micelles made of various lipids such as cholesterol, phospholipids, fatty acids, and derivatives thereof. Various modified lipids can also be used. Liposomes can reduce the toxicity of the active compounds, and increase their stability. Methods for preparing liposomal suspensions containing active ingredients therein are generally known in the art. See, e.g., U.S. Pat. No. 4,522,811; Prescott, Ed., Methods in Cell Biology, Volume XIV, Academic Press, New York, N.Y. (1976).

The active compounds can also be administered in combination with another active agent that synergistically treats or prevents the same symptoms or is effective for another disease or symptom in the patient treated so long as the other active agent does not interfere with or adversely affect the effects of the active compounds of this invention. Such other active agents include but are not limited to anti-inflammation agents, antiviral agents, antibiotics, antifungal agents, antithrombotic agents, cardiovascular drugs, cholesterol lowering agents, anti-cancer drugs, hypertension drugs, and the like.

### EXAMPLES

Synthesis of compounds of Formulae I-III can be accomplished according to the following general synthetic route. See Tables 1-3 for representative structures and relevant characterization data.

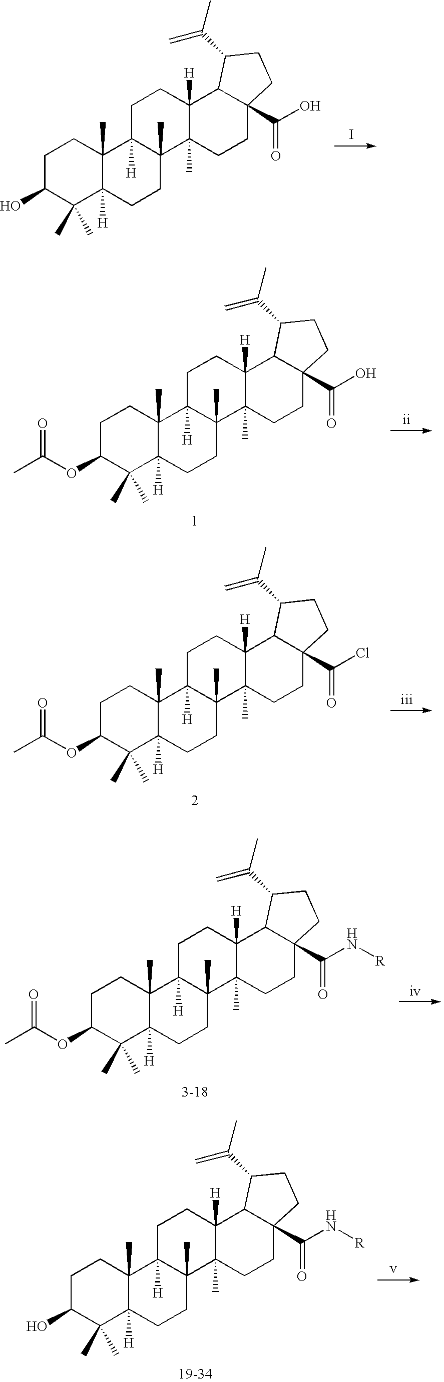

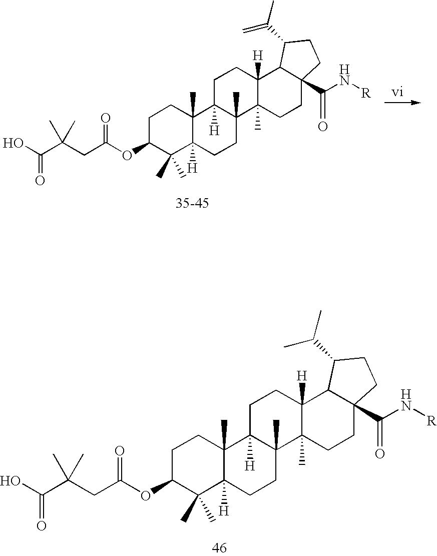

The above scheme summarizes the synthetic routes to the compounds in Tables 1-3 where the reagents/conditions are: i. Ac₂O, DMAP, Py, Δ. ii. Oxalyl Chloride (2M), CH₂Cl₂. iii. NHR₁R₂, TEA, CH₂Cl₂. iv. NaOH (4M), THF/MeOH. v. 2,2-Dimethylsuccinic anhydride, DMAP, Py, Δ. vi. PtO₂, H₂ (15 psi), AcOH.

General procedure for HPLC purification: Samples were dissolved in DMSO (˜50 mg/mL), and purified on a Phenomenex Synergi Hydro-RP (00G-4376-PO) HPLC column (250×21.2 mm, 10μ sphere size, 80 Å pore size), The solvent system is 50-90% acetonitrile in water (0.01% trifluoroacetic acid), run isocrally for up 25 minutes. Fraccion collection is based on absorption at 203λ.

### 3-O-acetyl-betulinic acid (1)

A solution of betulinic acid (0.50 g, 1.1 mmol) in anhydrous pyridine (10 mL) under nitrogen atmosphere was treated with Ac₂O (0.26 ml, 2.8 mmol) and DMAP (0.14 g, 1.1 mmol) and the mixture was refluxed for 1 h. The reaction mixture was diluted with CHCl₃ and washed with water. The organic layer was dried over MgSO₄ and concentrated under reduced pressure to give 1 (0.42 g, 76%).

¹H NMR (DMSO-d₆, 400 MHz) δ 0.79 (s, 6H, CH₃), 0.80 (s, 3H, CH₃), 0.87 (s, 3H, CH₃), 0.94 (s, 3H, CH₃), 1.25-1.62 (m, 18H, CH₂), 1.65 (s, 3H, CH₃), 1.75-1.85 (m, 2H, CH₂), 1.99 (s, 3H, CH₃CO), 2.08-2.14 (m, 1H), 2.18-2.27 (m, 1H), 2.90-3.00 (m, 1H), 4.36 (dd, 1H, J=11.24 Hz, J=4.8 Hz, H-3), 4.56 (m, 1H, CH═), 4.69 (d, 1H, J=2.15 Hz, CH═), 12.10 (bs, 1H, CO₂H).

### Preparation of the Acid Chlorides of 3-O-acetyl-betulinic acid (2)

Oxalyl chloride solution (2M in CH₂Cl₂, 4 mL) was added to the 3-O-acetyl-betulinic acid (0.1 g, 0.2 mmol) and stirred for 2 h. The mixture was concentrated to dryness under reduced pressure. The residue was diluted with dry CH₂Cl₂ (3×1 mL), concentrated to dryness under reduced pressure, and used without further purification.

### General Procedure for Synthesizing Compounds (3-18)

To a solution of the acid chloride 2 (0.2 mmol) in dry CH₂Cl₂ (5 mL) under nitrogen atmosphere was added the appropriate amine (0.26 mmol) and TEA (0.44 mmol, 0.061 mL). The reaction mixture was stirred at room temperature overnight, diluted with CH₂Cl₂ and then the CH₂Cl₂ layer washed with H₂O. The organic layer was dried over MgSO₄ and concentrated under reduced pressure to give the amide compound. In some cases the products were pure enough to use them directly for the next step, and some products were purified by HPLC.

TABLE 1

| Compound | | NMR (DMSO-d₆, 400 MHz). |
|---|---|---|
| No. | Structure | LC-MS (ESI) |
| 3 | 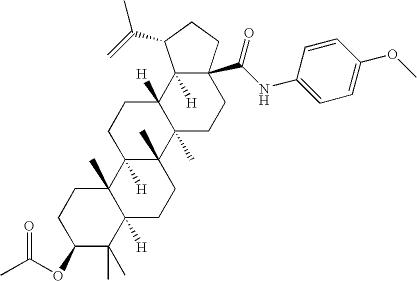 | 0.78 (s, 6 H, CH₃), 0.81 (s, 3 H, CH₃), 0.86 (s, 3 H, CH₃), 0.96 (s, 3 H, CH₃), 1.00-1.85 (m, 25 H), 1.99 (s, 3 H, CH₃CO), 2.30-2.40 (m, 1 H), 2.70 (m, 1 H), 3.20-3.00 (m, 2 H), 3.70 (s, 3 H, OCH₃), 4.36 (dd, 1 H, J = 11.6 Hz, J = 4.8 Hz, H-3), 4.55 (bs, 1 H, CH═), 4.68 (bs, 1 H, CH═), 6.84 (d, 2 H, J = 9.1, CH Arom), 7.45 (d, 2 H, J = 9.1 Hz, CH Arom), 9.29 (s, 1 H, NH). 604.54 (M + H)⁺. |
| 4 | 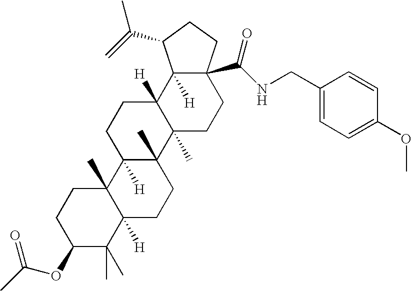 | 0.79 (s, 12 H, CH₃), 0.80 (s, 3 H, CH₃), 0.92 (s, 3 H, CH₃), 1.00-1.85 (m, 23 H, CH₂, CH₃), 1.99 (s, 3 H, CH₃CO), 2.10- 2.20 (m, 1 H), 2.95-3.05 (m, 1 H), 3.72 (s, 3 H, CH₃O), 4.10 (dd, 1 H, J = 15.0 Hz, J = 6.0 Hz, CH₂N), 4.22 (dd, 1 H, J = 15.0 Hz, J = 6.0 Hz, CH₂N), 4.36 (dd, 1 H, J = 10.8 Hz, J = 5.0 Hz, H-3), 4.53 (bs, 1 H, CH═), 4.65 (bs, 1 H, CH═), 6.84 (d, 2 H, J = 8.8 Hz, CH Arom), 7.15 (d, 2 H, J = 8.8 Hz, CH Arom), 8.09 (t, 1 H, J = 6.2 Hz, NH). 618.49 (M + H)⁺. |
| 5 | 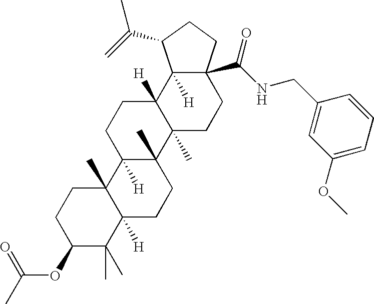 | 0.79 (s, 15 H, CH₃), 0.92 (s, 3 H, CH₃), 1.00-1.90 (m, 23 H), 1.99 (s, 3 H, CH₃CO), 2.10-2.25 (m, 1 H), 2.95-3.05 (m, 1 H), 3.72 (s, 3 H, CH₃O), 4.17 (dd, 1 H, J = 15.2 Hz, J = 5.9 Hz, CH₂N), 4.25 (dd, 1 H, J = 15.2 Hz, J = 5.3 Hz, CH₂N), 4.30-4.40 (m, 1 H, H-3), 4.53 (bs, 1 H, CH═), 4.65 (bs, 1 H, CH═), 6.70-6.85 (m, 3 H, CH Arom), 7.20 (t, 1 H, J = 7.7 Hz, CH Arom), 8.16 (bs, 1 H, NH). 618.30 (M + H)⁺. |
| 6 | 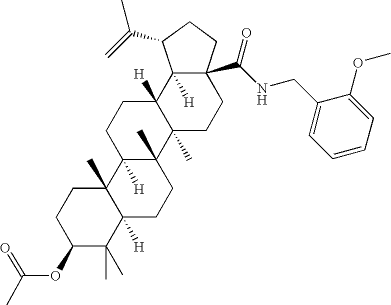 | 0.79 (s, 12 H, CH₃), 0.93 (s, 3 H, CH₃), 1.00-1.95 (m, 26 H), 1.99 (s, 3 H, CH₃CO), 2.20-2.30 (m, 1 H), 2.955-3.05 (m, 1 H), 3.79 (s, 3 H, CH₃O), 4.20 (d, 2 H, J = 5.8 Hz, CH₂N), 4.36 (dd, 1 H, J = 11.1 Hz, J = 4.8 Hz, H-3), 4.53 (bs, 1 H, CH═), 4.64 (d, 1 H, J = 2.5 Hz, CH═), 6.87 (dt, 1 H, J = 8.2 Hz, J = 0.8 Hz, CH Arom), 6.95 (d, 1 H, J = 8.2 Hz, CH Arom), 7.11 (d, 1 H, J = 6.1 Hz, CH Arom), 7.20 (dt, 1 H, J = 8.6 Hz, J = 1.6 Hz, CH Arom), 7.97 (t, 1 H, J = 5.8 Hz, NH). 618.4576 (M + H)⁺. |
| 7 | 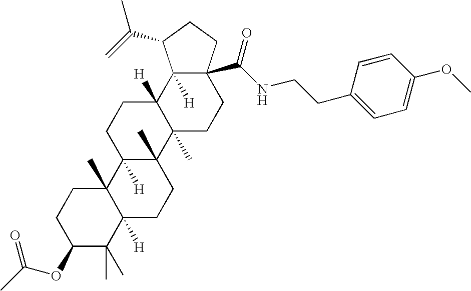 | 0.76 (s, 3 H, CH₃), 0.85 (s, 9 H, CH₃), 0.90-1.08 (m, 6 H), 1.09-1.75 (m, 23 H), 1.99 (s, 3 H, CH₃CO), 2.02-2.08 (m, 1 H), 2.56-2.76 (m, 2 H), 2.95-3.05 (m, 1 H, J = 11.0 Hz, J = 4.7 Hz), 3.10-3.16 (m, 1 H), 3.28-3.35 (m, 1 H), 3.70 (s, 3 H, CH₃O), 4.35 (dd, 1 H, J = 11.1 Hz, J = 4.8 Hz, H-3), 4.52 (bs, 1 H, CH═), 4.64 (d, 1 H, J = 2.5 Hz, CH═), 6.82 (d, 2 H, J = 8.61 Hz, CH Arom), 7.09 (d, 2 H, J = 8.6 Hz, CH Arom), 7.57 (t, 1 H, J = 5.4 Hz, NH). |
| 8 | 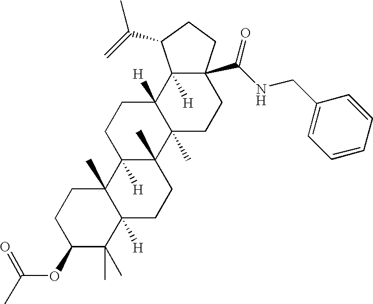 | 0.79 (s, 9 H, CH₃), 0.80 (s, 3 H, CH₃), 0.92 (s, 3 H, CH₃), 1.00-1.85 (m, 26 H), 1.99 (s, 3 H, CH₃CO), 2.15-2.25 (m, 1 H), 2.97- 3.15 (m, 1 H), 4.17 (dd, 1 H, J = 15.0 Hz, J = 6.2 Hz, CH₂N), 4.25-4.40 (m, 2 H, H-3 and CH₂N), 4.53 (bs, 1 H, CH═), 4.65 (d, 1 H, J = 2.5 Hz, CH═), 7.18-7.32 (m, 5 H, CH Arom), 8.17 (t, 1 H, J = 6.1 Hz, NH). 588.55 (M + H)⁺. |
| 9 | 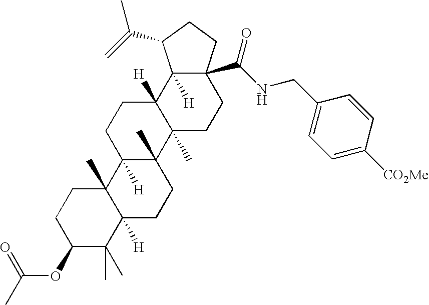 | 0.77 (s, 3 H, CH₃), 0.79 (s, 9 H, CH₃), 0.92 (s, 3 H, CH₃), 1.00-1.90 (m, 26 H), 1.99 (s, 3 H, CH₃CO), 2.15-2.25 (m, 1 H), 2.95- 3.05 (m, 1 H), 3.84 (s, 3 H, CO₂Me), 4.24 (dd, 1 H, J = 16.05 Hz, J = 6.0 Hz, CH₂N), 4.30-4.40 (m, 2 H, H-3 and CH₂N), 4.53 (bs, 1 H, CH═), 4.64 (d, 1 H, J = 2.1 Hz, CH═), 7.37 (d, 2 H, J = 8.4 Hz, CH Arom), 7.89 (d, 2 H, J = 8.4 Hz, CH Arom), 8.27 (t, 1 H, J = 6.0 Hz, NH). 646.54 (M + H)⁺. |
| 10 | 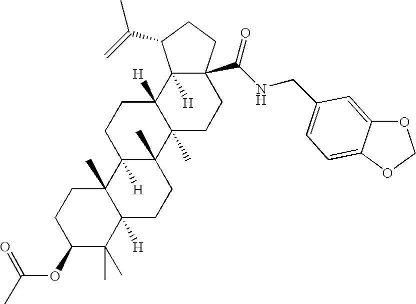 | 0.79 (s, 9 H, CH₃), 0.80 (s, 3 H, CH₃), 0.92 (s, 3 H, CH₃), 1.00-1.85 (m, 26 H), 1.99 (s, 3 H, CH₃CO), 2.10-2.20 (m, 1 H), 2.95- 3.08 (m, 1 H), 4.06 (dd, 1 H, J = 14.7 Hz, J = 6.1 Hz, CH₂N), 4.20 (dd, 1 H, J = 14.7 Hz, J = 6.0 Hz, CH₂N), 4.36 (dd, 1 H, J = 11.1 Hz, J = 4.8 Hz, H-3), 4.53 (bs, 1 H, CH═), 4.65 (d, 1 H, J = 2.3 Hz, CH═), 5.96 (d, 2 H, J = 2.2 Hz, OCH₂O), 6.70 (dd, 1 H, J = 7.8 Hz, J = 1.5 Hz, CH Arom), 6.78 (d, 1 H, J = 1.5 Hz, CH Arom), 6.82 (d, 1 H, J = 7.8 Hz, CH Arom), 8.10 (t, 1 H, J = 5.7 Hz, NH). 632.46 (M + H)⁺. |
| 11 | 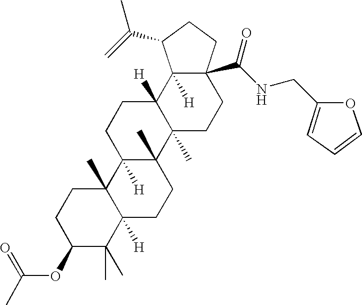 | 0.79 (s, 9 H, CH₃), 0.80 (s, 3 H, CH₃), 0.92 (s, 3 H, CH₃), 0.95-1.85 (m, 26 H), 1.99 (s, 3 H, CH₃CO), 2.10-2.20 (m, 1 H), 2.95- 3.08 (m, 1 H), 4.13 (dd, 1 H, J = 16.1 Hz, J = 5.6 Hz, CH₂N), 4.29 (dd, 1 H, J = 16.1 Hz, J = 5.9 Hz, CH₂N), 4.36 (dd, 1 H, J = 11.0 Hz, J = 4.9 Hz, H-3), 4.53 (bs, 1 H, CH═), 4.65 (bs, 1 H, CH═), 6.12 (dd, 1 H, J = 3.2 Hz, J = 0.9 Hz, CH Arom), 6.37 (dd, 1 H, J = 3.2 Hz, J = 1.9 Hz, CH Arom), 7.52 (dd, 1 H, J = 1.9 Hz, J = 0.9 Hz, CH Arom), 8.08 (t, 1 H, J = 5.9 Hz, NH). 578.41 (M + H)⁺. |
| 12 | 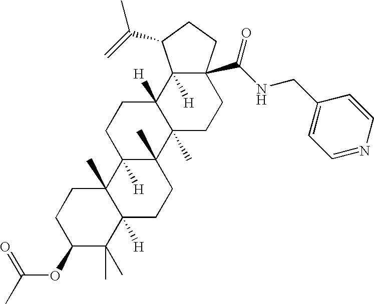 | 0.75 (s, 3 H, CH₃), 0.79 (s, 9 H, CH₃), 0.93 (s, 3 H, CH₃), 1.00-1.90 (m, 26 H), 1.99 (s, 3 H, CH₃CO), 2.15-2.25 (m, 1 H), 2.90- 3.06 (m, 1 H), 4.22 (dd, 1 H, J = 14.6 Hz, J = 6.4 Hz, CH₂N), 4.35-4.40 (m, 2 H, H-3 and CH₂N), 4.53 (bs, 1 H, CH═), 4.64 (bs, 1 H, CH═), 7.22 (d, 2 H, J = 5.9 Hz, CH Arom), 8.28 (bs, 1 H, NH), 8.47 (d, 2 H, J = 5.9 Hz, CH Arom). 589.71 (M + H)⁺. |
| 13 | 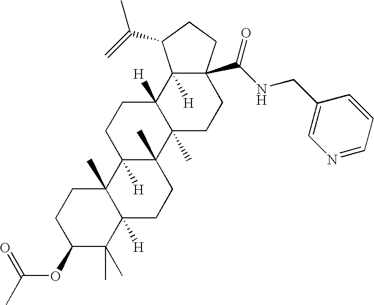 | 0.72 (s, 3 H, CH₃), 0.78 (s, 9 H, CH₃), 0.91 (s, 3 H, CH₃), 1.00-1.90 (m, 26 H), 1.99 (s, 3 H, CH₃CO), 2.10-2.20 (m, 1 H), 2.95- 3.06 (m, 1 H), 4.16 (dd, 1 H, J = 15.3 Hz, J = 6.0 Hz, CH₂N), 4.30 (dd, 1 H, J = 15.3 Hz, J = 5.9 Hz, CH₂N), 4.35 (dd, 1 H, J = 11.3 Hz, J = 4.8 Hz, H-3), 4.53 (bs, 1 H, CH═), 4.65 (d, 1 H, J = 2.5 Hz, CH═), 7.30-7.38 (m, 1 H, CH Arom), 7.63 (dt, 1 H, J = 8.0 Hz, J = 1.8 Hz, CH Arom), 8.25 (t, 1 H, J = 6.1 Hz, NH), 8.42 (dd, 1 H, J = 4.7 Hz, J = 1.8 Hz, CH Arom), 8.46 (d, 1 H, J = 1.8 Hz, CH Arom). 589.4372 (M + H)⁺. |
| 14 | 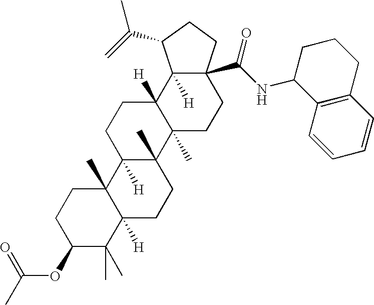 | 0.80 (s, 9 H, CH₃), 0.84 (s, 3 H, CH₃), 0.93 (s, 3 H, CH₃), 0.95 (s, 3 H, CH₃), 0.98 (s, 3 H, CH₃), 1.00-1.95 (m, 28 H), 2.00 (s, 3 H, CH₃CO), 2.15-2.25 (m, 1 H), 2.60- 2.80 (m, 4 H), 3.00-3.15 (m, 1 H), 4.30- 4.40 (m, 1 H, H-3), 4.55 (bs, 1 H, CH═), 4.65-4.70 (m, 1 H, CH═), 4.95-5.10 (m, 1 H, CHN), 7.00-7.20 (m, 4 H, CH Arom), 7.80-7.90 (m, 1 H, NH). 628.4716 (M + H)⁺. |
| 15 | 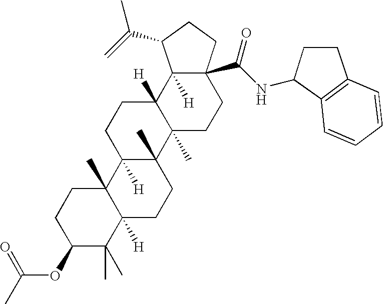 | 0.79 (s, 6 H, CH₃), 0.84 (s, 3 H, CH₃), 0.88 (s, 3 H, CH₃), 0.94 (s, 3 H, CH₃), 0.95 (s, 3 H, CH₃), 0.97 (s, 3 H, CH₃), 1.00-1.95 (m, 20 H), 1.99 (s, 3 H, CH₃CO), 2.25-2.35 (m, 1 H), 2.55-2.80 (m, 4 H), 3.00-3.20 (m, 1 H), 4.33-4.42 (m, 1 H, H-3), 4.55 (bs, 1 H, CH═), 4.65-4.72 (m, 1 H, CH═), 5.25- 5.40 (m, 1 H, CHN), 7.05-7.35 (m, 4 H, CH Arom), 7.85 (t, 1 H, J = 8.02 Hz, NH). 614.4557 (M + H)⁺. |
| 16 | 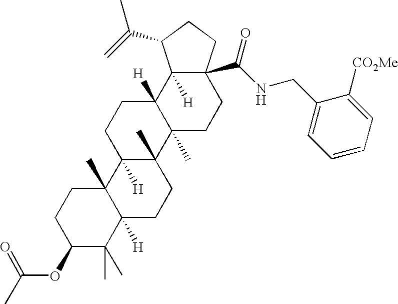 | 0.75 (s, 3 H, CH₃), 0.79 (s, 9 H, CH₃), 0.93 (s, 3 H, CH₃), 1.00-1.95 (m, 26 H), 1.99 (s, 3 H, CH₃CO), 2.15-2.25 (m, 1 H), 2.90- 3.05 (m, 1 H), 3.85 (s, 3 H, CH₃O), 4.35- 4.40 (m, 2 H, H-3 and CH₂N), 4.53 (m, 2 H, CH═ and CH₂N)), 4.64 (bs, 1 H, CH═), 7.30-7.70 (m, 3 H, CH Arom), 7.84 (d, 1 H, J = 6.5 Hz, CH Arom), 8.08 (t, 1 H, J = 5.3 Hz, NH). 668.52 (M + H)⁺. |
| 17 | 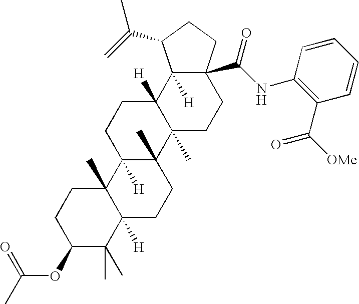 | 0.78 (s, 6 H, CH₃), 0.80 (s, 3 H, CH₃), 0.84 (s, 3 H, CH₃), 0.98 (s, 3 H, CH₃), 1.10-1.95 (m, 26 H), 1.99 (s, 3 H, CH₃CO), 2.25-2.15 (m, 1 H), 3.00-3.15 (m, 1 H), 3.87 (s, 3 H, OCH₃), 4.36 (dd, 1 H, J = 11.0 Hz, J = 5.1 Hz, H-3), 4.58 (bs, 1 H, CH═), 4.71 (bs, 1 H, CH═), 7.14-7.18 (m, 1 H, CH Arom), 7.59-7.63 (m, 1 H, CH Arom), 7.96 (dd, 1 H, J = 8.0 Hz, J = 1.6 Hz, CH Arom), 8.42 (d, 1 H, J = 7.4 Hz, CH Arom), 10.91 (s, 1 H, NH). 630.41640 (M − H)⁻. |
| 18 | 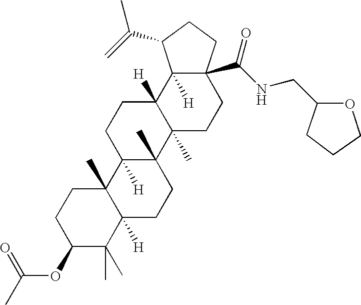 | 0.78 (s, 9 H, CH₃), 0.84 (s, 3 H, CH₃), 0.92 (s, 3 H, CH₃), 1.00-1.90 (m, 30 H), 1.99 (s, 3 H, CH₃CO), 2.10-2.20 (m, 1 H), 2.90- 3.05 (m, 3 H), 3.10-3.25 (m, 1 H), 3.54- 3.62 (m, 1 H, OCH), 3.68-3.76 (m, 1 H, OCH), 3.70-3.88 (m, 1 H, OCH), 4.30- 4.40 (m, 1 H, H-3), 4.53 (bs, 1 H, CH═), 4.65 (bs, 1 H, CH═), 7.62 (bs, 1 H, NH). 582.4537 (M + H)⁺. |

### General Procedure for Synthesizing Compounds (19-34)

A solution of the appropriate amide (0.21 mmol) in THF (1.6 mL) and Methanol (1 mL) was treated with NaOH (4M, 0.27 mL). The mixture was stirred at room temperature overnight, and then the solvents were evaporated under reduced pressure. The residue was diluted with CH₂Cl₂ and washed with a HCl solution (0.5 N). The organic layer was dried over MgSO₄ and concentrated under reduced pressure to give the amide compound 19-34.

TABLE 2

| Compound | | NMR (DMSO-d₆, 400 MHz) |
|---|---|---|
| No. | Structure | LC-MS (ESI) |
| 19 | 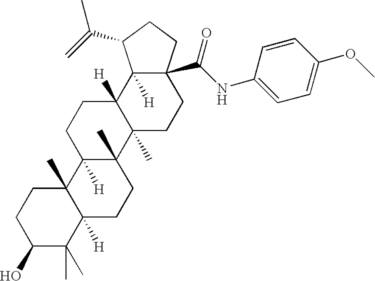 | 0.64 (s, 3 H, CH₃), 0.76 (s, 3 H, CH₃), 0.85 (s, 3 H, CH₃), 0.86 (s, 3 H, CH₃), 0.94 (s, 3 H, CH₃), 1.00-2.05 (m, 24 H), 2.40-2.30 (m, 1 H), 2.65-2.75 (m, 1 H), 2.90-3.10 (m, 2 H, CH₂), 3.55-3.65 (m, 1 H), 3.71 (s, 3 H, OCH₃), 4.28 (d, 1 H, J = 5.29 Hz, H-3), 4.55 (bs, 1 H, CH═), 4.68 (d, 1 H, J = 2.5 Hz, CH═), 6.84 (d, 2 H, J = 9.1, CH Arom), 7.45 (d, 2 H, J = 9.1 Hz, CH Arom), 9.29 (s, 1 H, NH). 562.55 (M + H)⁺. |
| 20 | 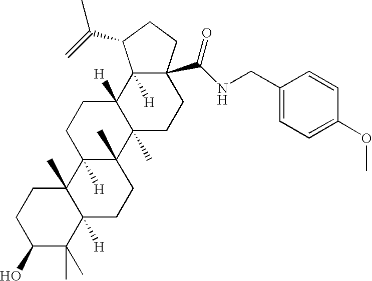 | 0.65 (s, 3 H, CH₃), 0.76 (s, 3 H, CH₃), 0.78 (s, 3 H, CH₃), 0.86 (s, 3 H, CH₃), 0.90 (s, 3 H, CH₃), 0.95-1.85 (m, 27 H), 2.10-2.20 (m, 1 H), 2.90-3.10 (m, 2 H), 3.72 (s, 3 H, CH₃O), 4.10 (dd, 1 H, J = 14.8 Hz, J = 5.9 Hz, CH₂N), 4.21 (dd, 1 H, J = 14.8 Hz, J = 6.0 Hz, CH₂N), 4.27 (d, 1 H, J = 5.1 Hz, H-3), 4.53 (bs, 1 H, CH═), 4.64 (bs, 1 H, CH═), 6.84 (d, 2 H, J = 8.7 Hz, CH Arom), 7.15 (d, 2 H, J = 8.7 Hz, CH Arom), 8.09 (t, 1 H, J = 6.1 Hz, NH). 576.4390 (M + H)⁺. |
| 21 | 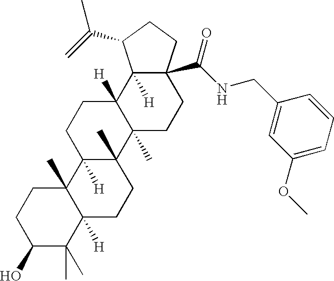 | 0.65 (s, 3 H, CH₃), 0.75 (s, 3 H, CH₃), 0.77 (s, 3 H, CH₃), 0.86 (s, 3 H, CH₃), 0.91 (s, 3 H, CH₃), 1.00-1.90 (m, 26 H), 2.15-2.25 (m, 1 H), 2.92-3.05 (m, 2 H), 3.71 (s, 3 H, CH₃O), 4.17 (dd, 1 H, J = 15.3 Hz, J = 5.9 Hz, CH₂N), 4.25 (dd, 1 H, J = 15.3 Hz, J = 6.1 Hz, CH₂N), 4.27 (d, 1 H, J = 5.1 Hz, H-3), 4.53 (bs, 1 H, CH═), 4.65 (d, 1 H, J = 2.3 Hz, CH═), 6.74-6.84 (m, 3 H, CH Arom), 7.19 (t, 1 H, J = 7.7 Hz, CH Arom), 8.15 (t, 1 H, J = 6.1 Hz, NH). 576.53 (M + H)⁺. |
| 22 | 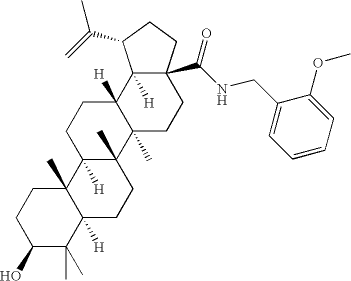 | 0.65 (s, 3 H, CH₃), 0.75 (s, 3 H, CH₃), 0.77 (s, 3 H, CH₃), 0.87 (s, 3 H, CH₃), 0.91 (s, 3 H, CH₃), 1.00-1.90 (m, 26 H), 2.25-2.35 (m, 1 H), 2.90-3.05 (m, 2 H), 3.79 (s, 3 H, CH₃O), 4.20 (d, 2 H, J = 5.8 Hz, CH₂N), 4.27 (d, 1 H, J = 5.1 Hz, H- 3), 4.52 (bs, 1 H, CH═), 4.64 (d, 1 H, J = 2.5 Hz, CH═), 6.86 (dt, 1 H, J = 7.5 Hz, J = 1.0 Hz, CH Arom), 6.95 (d, 1 H, J = 7.43 Hz, CH Arom), 7.11 (d, 1 H, J = 5.9 Hz, CH Arom), 7.19 (dt, 1 H, J = 8.6 Hz, J = 1.6 Hz, CH Arom), 7.97 (t, 1 H, J = 5.8 Hz, NH). |
| 23 | 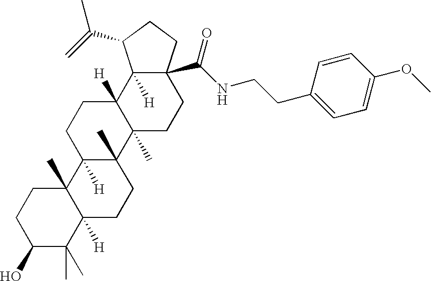 | 0.64 (s, 3 H, CH₃), 0.75 (s, 6 H, CH₃), 0.86 (s, 3 H, CH₃), 0.88 (s, 3 H, CH₃), 1.00-1.80 (m, 27 H), 2.00-2.10 (m, 1 H), 2.55-2.70 (m, 2 H), 2.90-3.05 (m, 2 H), 3.10-3.20 (m, 1 H), 3.70 (s, 3 H, CH₃O), 4.27 (d, 1 H, J = 5.1 Hz, H-3), 4.52 (bs, 1 H, CH═), 4.63 (d, 1 H, J = 2.0 Hz, CH═), 6.82 (d, 2 H, J = 8.7 Hz, CH Arom), 7.10 (d, 2 H, J = 8.7 Hz, CH Arom), 7.57 (t, 1 H, J = 5.9 Hz, NH). 590.53 (M + H)⁺. |
| 24 | 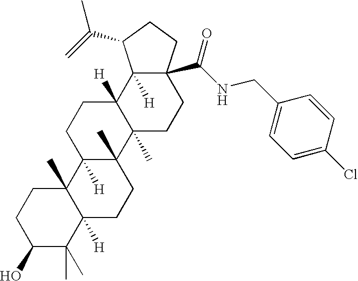 | 0.65 (s, 3 H, CH₃), 0.74 (s, 3 H, CH₃), 0.75 (s, 3 H, CH₃), 0.86 (s, 3 H, CH₃), 0.90 (s, 3 H, CH₃), 0.95-1.85 (m, 26 H), 2.10-2.25 (m, 1 H), 2.90-3.07 (m, 2 H), 4.15 (dd, 1 H, J = 15.0 Hz, J = 6.1 Hz, CH₂N), 4.25 (dd, 1 H, J = 15.0 Hz, J = 5.7 Hz, CH₂N), 4.27 (d, 1 H, J = 5.3 Hz, H-3), 4.53 (bs, 1 H, CH═), 4.64 (d, 1 H, J = 2.1 Hz, CH═), 7.25 (d, 2 H, J = 8.5 Hz, CH Arom), 7.35 (d, 2 H, J = 8.5 Hz, CH Arom), 8.20 (t, 1 H, J = 5.9 Hz, NH). 580.58 (M + H)⁺. |
| 25 | 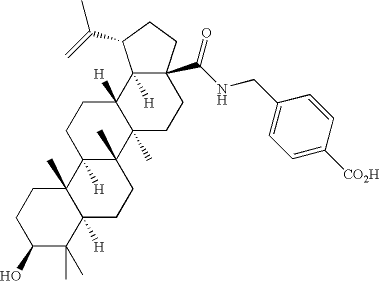 | 0.65 (s, 3 H, CH₃), 0.75 (s, 3 H, CH₃), 0.77 (s, 3 H, CH₃), 0.86 (s, 3 H, CH₃), 0.91 (s, 3 H, CH₃), 1.00-1.90 (m, 26 H), 2.15-2.25 (m, 1 H), 2.90-3.07 (m, 2 H), 4.22 (dd, 1 H, J = 15.6 Hz, J = 5.9 Hz, CH₂N), 4.27 (d, 1 H, J = 5.1 Hz, H-3), 4.34 (dd, 1 H, J = 15.6 Hz, J = 6.0 Hz, CH₂N), 4.53 (bs, 1 H, CH═), 4.64 (bs, 1 H, CH═), 7.34 (d, 2 H, J = 8.1 Hz, CH Arom), 7.86 (d, 2 H, J = 8.1 Hz, CH Arom), 8.25 (t, 1 H, J = 6.0 Hz, NH), 12.87 (bs, 1 H, CO₂H). 588.4057 (M − H)⁻. |
| 26 | 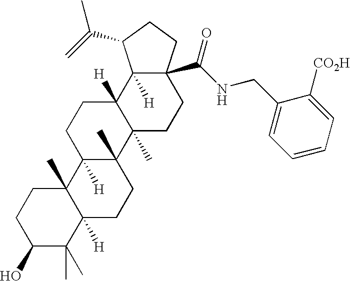 | 0.64 (s, 3 H, CH₃), 0.66 (s, 3 H, CH₃), 0.73 (s, 3 H, CH₃), 0.86 (s, 3 H, CH₃), 0.89 (s, 3 H, CH₃), 0.90-1.85 (m, 26 H), 2.05-2.15 (m, 1 H), 2.85-3.08 (m, 2 H), 4.27 (bs, 1 H, H-3), 4.44 (d, 2 H, J = 5.5 Hz, CH₂N), 4.52 (bs, 1 H, CH═), 4.64 (bs, 1 H, CH═), 7.21 (t, 2 H, J = 6.6 Hz, CH Arom), 7.29 (t, 1 H, J = 6.8 Hz, CH Arom), 7.71 (d, 1 H, J = 7.2 Hz, CH Arom), 8.48 (bs, 1 H, NH). 590.4197 (M + H)⁺. |
| 27 | 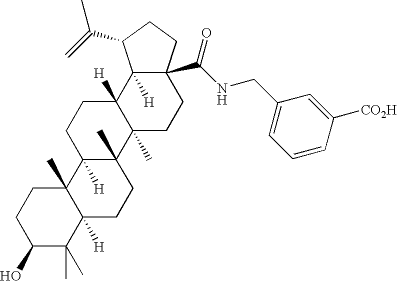 | 0.64 (s, 3 H, CH₃), 0.70 (s, 3 H, CH₃), 0.73 (s, 3 H, CH₃), 0.86 (s, 3 H, CH₃), 0.90 (s, 3 H, CH₃), 1.00-1.80 (m, 26 H), 2.15-2.25 (m, 1 H), 2.90-3.08 (m, 2 H), 4.20 (dd, 1 H, J = 15.0 Hz, J = 5.6 Hz, CH₂N), 4.27 (d, 1 H, J = 4.9 Hz, H-3), 4.34 (dd, 1 H, J = 15.0 Hz, J = 5.6 Hz, CH₂N), 4.53 (bs, 1 H, CH═), 4.64 (bs, 1 H, CH═), 7.41 (t, 1 H, J = 7.6 Hz, CH Arom), 7.47 (d, 1 H, J = 7.6 Hz, CH Arom), 7.78 (d, 1 H, J = 7.2 Hz, CH Arom), 7.85 (s, 1 H, CH Arom), 8.25 (t, 1 H, J = 5.7 Hz, NH), 12.88 (bs, 1 H, CO₂H). 590.4207 (M + H)⁺. |
| 28 | 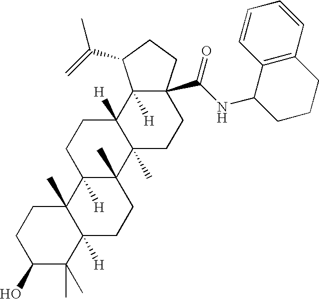 | 0.65 (s, 3 H, CH₃), 0.66 (s, 3 H, CH₃), 0.77 (s, 3 H, CH₃), 0.79 (s, 3 H, CH₃), 0.87 (s, 6 H, CH₃), 0.92 (s, 3 H, CH₃), 0.93 (s, 3 H, CH₃), 0.97 (s, 3 H, CH₃), 1.00-1.95 (m, 17 H), 2.15-2.27 (m, 1 H), 2.55-2.80 (m, 3 H), 2.80-3.17 (m, 2 H), 4.28 (dd, 1 H, J = 5.1 Hz, J = 2.3 Hz, H- 3), 4.55 (bs, 1 H, CH═), 4.67 (d, 1 H, J = 6.3 Hz, CH═), 4.95-5.10 (m, 1 H, CNH), 7.00-7.20 (m, 4 H, CH Arom), 7.80-7.80 (m, 1 H, NH). 589.63 (M + H)⁺. |
| 29 | 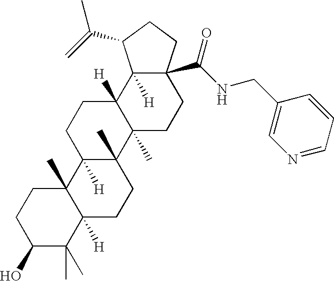 | 0.64 (s, 3 H, CH₃), 0.71 (s, 3 H, CH₃), 0.74 (s, 3 H, CH₃), 0.86 (s, 9 H, CH₃), 0.90 (s, 3 H, CH₃), 0.95-1.90 (m, 26 H), 2.10-2.20 (m, 1 H), 2.90-3.10 (m, 2 H), 4.16 (dd, 1 H, J = 15.0 Hz, J = 5.8 Hz, CH₂N), 4.27 (d, 1 H, J = 5.1 Hz, H-3), 4.29 (dd, 1 H, J = 15.0 Hz, J = 5.5 Hz, CH₂N), 4.53 (bs, 1 H, CH═), 4.64 (bs, 1 H, CH═), 7.30-7.36 (m, 1 H, CH Arom), 7.63 (dt, 1 H, J = 8.0 Hz, J = 1.6 Hz, CH Arom), 8.25 (t, 1 H, J = 5.9 Hz, NH), 8.42 (dd, 1 H, J = 4.7 Hz, J = 1.6 Hz, CH Arom), 8.46 (d, 1 H, J = 1.6 Hz, CH Arom). 547.66 (M + H)⁺. |
| 30 | 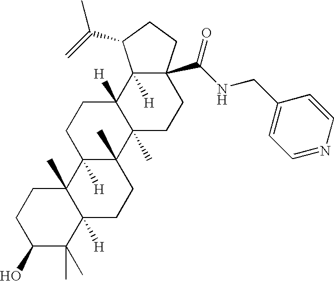 | 0.64 (s, 3 H, CH₃), 0.72 (s, 3 H, CH₃), 0.74 (s, 3 H, CH₃), 0.87 (s, 9 H, CH₃), 0.91 (s, 3 H, CH₃), 1.00-1.95 (m, 26 H), 2.15-2.25 (m, 1 H), 2.90-3.02 (m, 2 H), 4.36 (d, 2 H, J = 5.8 Hz CH₂N), 4.53 (bs, 1 H, CH═), 4.63 (d, 1 H, J = 2.1 Hz, CH═), 7.60 (d, 2 H, J = 6.3 Hz, CH Arom), 8.44 (t, 1 H, J = 5.8 Hz, NH), 8.71 (d, 2 H, J = 6.3 Hz, CH Arom). 547.4272 (M + H)⁺. |
| 31 | 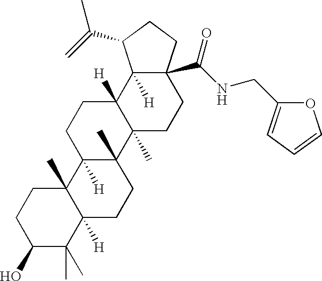 | 0.64 (s, 3 H, CH₃), 0.75 (s, 3 H, CH₃), 0.78 (s, 3 H, CH₃), 0.86 (s, 3 H, CH₃), 0.90 (s, 3 H, CH₃), 0.95-1.85 (m, 26 H), 2.10-2.20 (m, 1 H), 2.90-3.08 (m, 2 H), 4.13 (dd, 1 H, J = 15.7 Hz, J = 5.6 Hz, CH₂N), 4.25-4.33 (m, 2 H, H-3 and CH₂N), 4.53 (bs, 1 H, CH═), 4.65 (d, 1 H, J = 2.3 Hz, CH═), 6.11 (dd, 1 H, J = 3.1 Hz, J = 0.8 Hz, CH Arom), 6.37 (dd, 1 H, J = 3.1 Hz, J = 1.8 Hz, CH Arom), 7.52 (dd, 1 H, J = 1.8 Hz, J = 0.8 Hz, CH Arom), 8.07 (t, 1 H, J = 5.9 Hz, NH). 536.4100 (M + H)⁺. |
| 32 | 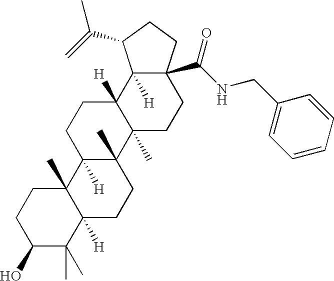 | 0.65 (s, 3 H, CH₃), 0.75 (s, 3 H, CH₃), 0.78 (s, 3 H, CH₃), 0.86 (s, 3 H, CH₃), 0.91 (s, 3 H, CH₃), 1.00-1.85 (m, 26 H), 2.14-2.25 (m, 1 H), 2.90-3.10 (m, 2 H), 4.16 (dd, 1 H, J = 15.2 Hz, J = 6.0 Hz, CH₂N), 4.25-4.35 (m, 2 H, H-3 and CH₂N), 4.53 (bs, 1 H, CH═), 4.65 (d, 1 H, J = 1.8 Hz, CH═), 7.16-7.34 (m, 5 H, CH Arom), 8.17 (t, 1 H, J = 5.9 Hz, NH). 546.57 (M + H)⁺. |
| 33 | 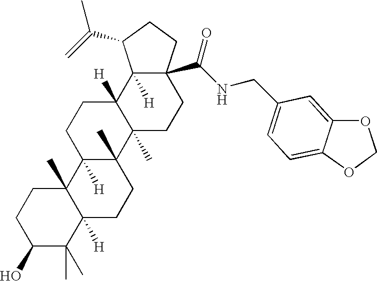 | 0.65 (s, 3 H, CH₃), 0.75 (s, 3 H, CH₃), 0.77 (s, 3 H, CH₃), 0.86 (s, 3 H, CH₃), 0.90 (s, 3 H, CH₃), 1.00-1.85 (m, 26 H), 2.10-2.20 (m, 1 H), 2.90-3.08 (m, 2 H), 4.06 (dd, 1 H, J = 15.0 Hz, J = 6.1 Hz, CH₂N), 4.20 (dd, 1 H, J = 15.0 Hz, J = 6.0 Hz, CH₂N), 4.27 (d, 1 H, J = 5.1 Hz, H-3), 4.53 (bs, 1 H, CH═), 4.65 (d, 1 H, J = 2.2 Hz, CH═), 5.96 (d, 2 H, J = 2.2 Hz, OCH₂O), 6.70 (dd, 1 H, J = 8.0 Hz, J = 1.7 Hz, CH Arom), 6.78 (d, 1 H, J = 1.7 Hz, CH Arom), 6.82 (d, 1 H, J = 8.0 Hz, CH Arom), 8.10 (t, 1 H, J = 5.9 Hz, NH). 590.46 (M + H)⁺. |
| 34 | 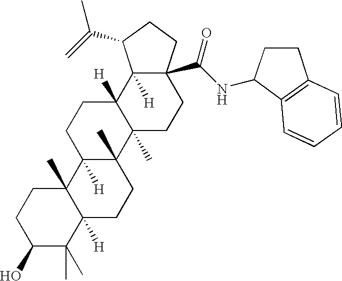 | 0.65 (s, 3 H, CH₃), 0.66 (s, 3 H, CH₃), 0.77 (s, 3 H, CH₃), 0.79 (s, 3 H, CH₃), 0.87 (s, 6 H, CH₃), 0.92 (s, 3 H, CH₃), 0.93 (s, 3 H, CH₃), 0.96 (s, 3 H, CH₃), 1.00-1.95 (m, 14 H), 2.05-2.35 (m, 2 H), 2.55-3.15 (m, 5 H), 4.26-4.30 (m, 1 H, H- 3), 4.55 (bs, 1 H, CH═), 4.72-4.65 (m, 1 H, CH═), 5.28-5.40 (m, 1 H, CNH), 7.04-7.26 (m, 4 H, CH Arom), 7.84 (m, 1 H, NH). 572.4455 (M + H)⁺. |

### General Procedure for Synthesizing Compounds (35-45)

A solution of the appropriate amide 19-34 (0.17 mmol) in dry Pyridine (4 mL), under nitrogen atmosphere, was treated with 2,2-Dimethylsuccinic anhydride (0.109 g, 0.85 mmol) and DMAP (0.021 g, 0.17 mmol) and the mixture was refluxed overnight. The reaction mixture was diluted with CH₂Cl₂ and washed with H₂O. The organic layer was dried over MgSO₄ and concentrated under reduced pressure to give the carboxylic acid product. The crude material was purified by HPLC.

TABLE 3

| Com- | | |
|---|---|---|
| pound | | NMR (DMSO-d₆, 400 MHz) |
| No. | Structure | LC-MS (ESI) |
| 35 | 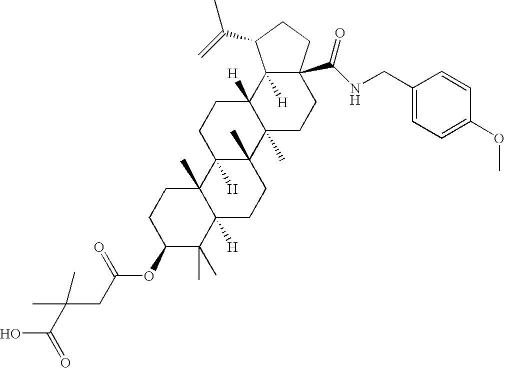 | 0.78 (s, 6 H, CH₃), 0.79 (s, 3 H, CH₃), 0.80 (s, 3 H, CH₃), 0.92 (s, 3 H, CH₃), 1.15 (s, 3 H, CH₃), 1.16 (s, 3 H, CH₃), 1.20-1.85 (m, 32 H), 2.12-2.20 (m, 1 H), 2.98-3.08 (m, 1 H), 3.72 (s, 3 H, CH₃O), 4.10 (dd, 1 H, J = 14.8 Hz, J = 6.3 Hz, CH₂N), 4.21 (dd, 1 H, J = 14.8 Hz, J = 5.9 Hz, CH₂N), 4.36 (dd, 1 H, J = 11.4 Hz, J = 5.0 Hz, H- 3), 4.53 (bs, 1 H, CH═), 4.65 (d, 1 H, J = 2.1 Hz, CH═), 6.84 (d, 2 H, J = 8.6 Hz, CH Arom), 7.15 (d, 2 H, J = 8.6 Hz, CH Arom), 8.09 (t, 1 H, J = 5.8 Hz, NH). 705.50 (M + H)⁺. |
| 36 | 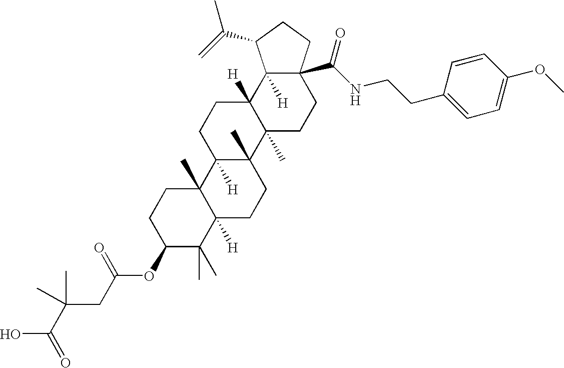 | 0.75 (s, 3 H, CH₃), 0.77 (s, 3 H, CH₃), 0.78 (s, 6 H, CH₃), 0.89 (s, 3 H, CH₃), 1.15 (s, 3 H, CH₃), 1.16 (s, 3 H, CH₃), 1.20-1.80 (m, 29 H), 2.00-2.10 (m, 1 H), 2.55-2.70 (m, 2 H), 2.90-3.02 (m, 1 H), 3.10-3.20 (m, 1 H), 3.70 (s, 3 H, CH₃O), 4.35 (dd, 1 H, J = 11.3 Hz, J = 4.5 Hz, H-3), 4.52 (bs, 1 H, CH═), 4.63 (bs, 1 H, CH═), 6.82 (d, 2 H, J = 8.6 Hz, CH Arom), 7.09 (d, 2 H, J = 8.6 Hz, CH Arom), 7.57 (t, 1 H, J = 5.5 Hz, NH), 12.17 (bs, 1 H, CO₂H). 718.58 (M + H)⁺. |
| 37 | 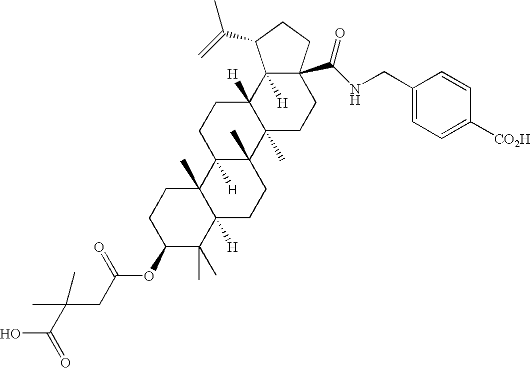 | 0.78 (s, 6 H, CH₃), 0.79 (s, 3 H, CH₃), 0.92 (s, 3 H, CH₃), 1.16 (s, 3 H, CH₃), 1.17 (s, 3 H, CH₃), 1.20-1.85 (m, 33 H), 2.12-2.25 (m, 1 H), 2.95-3.08 (m, 1 H), 4.22 (dd, 1 H, J = 15.4 Hz, J = 5.7 Hz, CH₂N), 4.30- 4.40 (m, 2 H, H-3 and CH₂N), 4.53 (bs, 1 H, CH═), 4.65 (bs, 1 H, CH═), 7.34 (d, 2 H, J = 8.3 Hz, CH Arom), 7.86 (d, 2 H, J = 8.3 Hz, CH Arom), 8.26 (t, 1 H, J = 5.7 Hz, NH). 718.55 (M + H)⁺. |
| 38 | 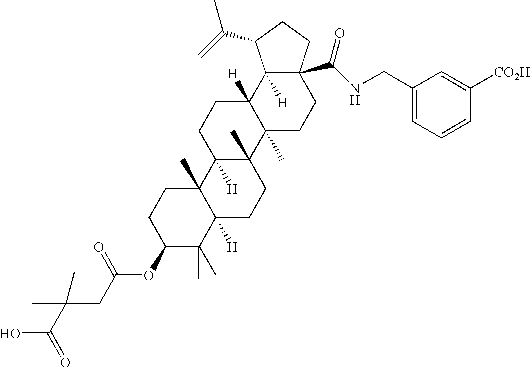 | 0.71 (s, 3 H, CH₃), 0.77 (s, 9 H, CH₃), 0.91 (s, 3 H, CH₃), 1.15 (s, 3 H, CH₃), 1.16 (s, 3 H, CH₃), 1.20-1.90 (m, 30 H), 2.12-2.25 (m, 1 H), 2.95-3.08 (m, 1 H), 4.20 (dd, 1 H, J = 15.3 Hz, J = 5.5 Hz, CH₂N), 4.29- 4.40 (m, 2 H, H-3 and CH₂N), 4.53 (bs, 1 H, CH═), 4.64 (bs, 1 H, CH═), 7.41 (t, 1 H, J = 7.5 Hz, CH Arom), 7.48 (d, 1 H, J = 7.6 Hz, CH Arom), 7.78 (d, 1 H, J = 7.4 Hz, CH Arom), 7.85 (bs, 1 H, CH Arom), 8.26 (t, 1 H, J = 5.8 Hz, NH). 718.4710 (M + H)⁺. |
| 39 | 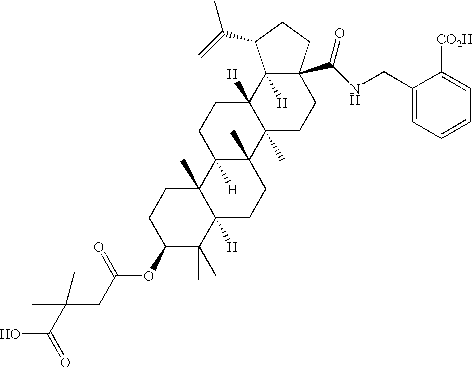 | 0.75 (s, 3 H, CH₃), 0.78 (s, 9 H, CH₃), 0.93 (s, 3 H, CH₃), 1.00-1.95 (m, 36 H), 2.12- 2.25 (m, 1 H), 2.95-3.08 (m, 1 H), 4.30- 4.40 (m, 1 H, H-3), 4.45-4.60 (m, 3 H, CH═ and CH₂N), 4.63 (bs, 1 H, CH═), 7.30-7.40 (m, 2 H, CH Arom), 7.50 (m, 1 H, CH Arom), 7.85 (d, 1 H, J = 7.4 Hz, CH Arom), 8.08 (bs, 1 H, NH). 716.59 (M − H)⁻. |
| 40 | 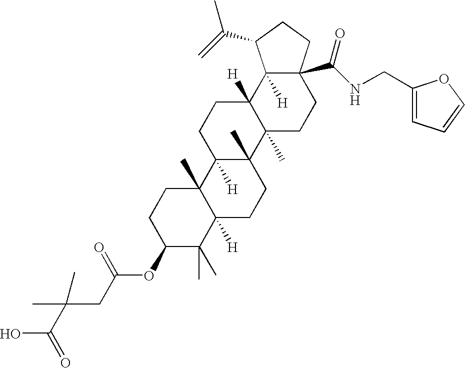 | 0.78 (s, 6 H, CH₃), 0.79 (s, 6 H, CH₃), 0.91 (s, 3 H, CH₃), 1.05-1.85 (m, 34 H), 2.10- 2.20 (m, 1 H), 2.95-3.05 (m, 1 H), 4.13 (dd, 1 H, J = 16.1 Hz, J = 5.8 Hz, CH₂N), 4.25-4.40 (m, 2 H, H-3 and CH₂N), 4.53 (bs, 1 H, CH═), 4.65 (bs, 1 H, CH═), 6.11 (dd, 1 H, J = 3.0 Hz, J = 0.7 Hz, CH Arom), 6.37 (dd, 1 H, J = 3.0 Hz, J = 1.9 Hz, CH Arom), 7.52 (dd, 1 H, J = 1.9 Hz, J = 0.7 Hz, CH Arom), 8.08 (t, 1 H, J = 5.9 Hz, NH), 12.1 (bs, 1 H, CO₂H). 664.49 (M + H)⁺. |
| 41 | 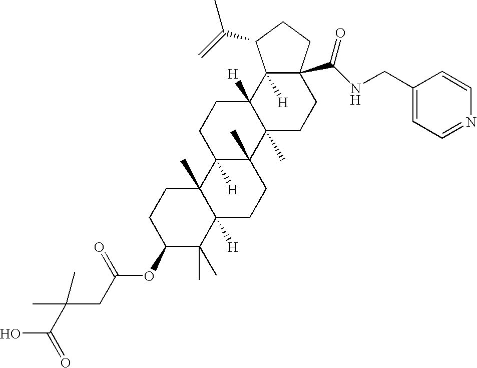 | 0.75 (s, 3 H, CH₃), 0.78 (s, 9 H, CH₃), 0.92 (s, 3 H, CH₃), 1.15 (s, 3 H, CH₃), 1.16 (s, 3 H, CH₃), 1.20-1.85 (m, 28 H), 2.12-2.25 (m, 1 H), 2.95-3.08 (m, 1 H), 4.19 (dd, 1 H, J = 16.1 Hz, J = 6.1 Hz, CH₂N), 4.26 (dd, 1 H, J = 16.1 Hz, J = 5.8 Hz, CH₂N), 4.36 (dd, 1 H, J = 11.5 Hz, J = 4.9 Hz, H-3), 4.53 (bs, 1 H, CH═), 4.65 (bs, 1 H, CH═), 7.22 (d, 2 H, J = 6.0 Hz, CH Arom), 8.29 (t, 1 H, J = 5.5 Hz, NH), 8.47 (d, 2 H, J = 6.0 Hz, CH Arom), 12.17 (bs, 1 H, CO₂H). 675.67 (M + H)⁺. |
| 42 | 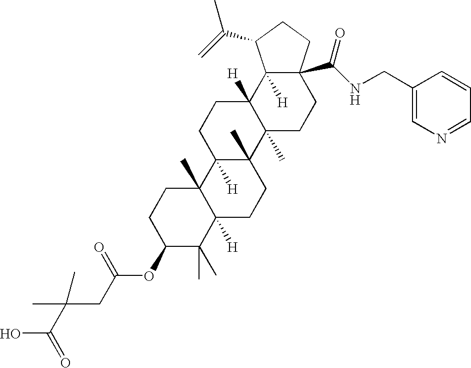 | 0.68 (s, 3 H, CH₃), 0.77 (s, 9 H, CH₃), 0.91 (s, 3 H, CH₃), 1.15 (s, 3 H, CH₃), 1.16 (s, 3 H, CH₃), 1.20-1.85 (m, 29 H), 2.12-2.20 (m, 1 H), 2.95-3.05 (m, 1 H), 4.22 (dd, 1 H, J = 15.1 Hz, J = 5.7 Hz, CH₂N), 4.30- 4.40 (m, 2 H, H-3 and CH₂N), 4.53 (bs, 1 H, CH═), 4.64 (bs, 1 H, CH═), 7.55-7.60 (m, 1 H, CH Arom), 7.88-7.94 (m, 1 H, CH Arom), 8.32 (bs, 1 H, NH), 8.55-8.60 (m, 2 H, CH Arom). 675.4873 (M + H)⁺. |
| 43 | 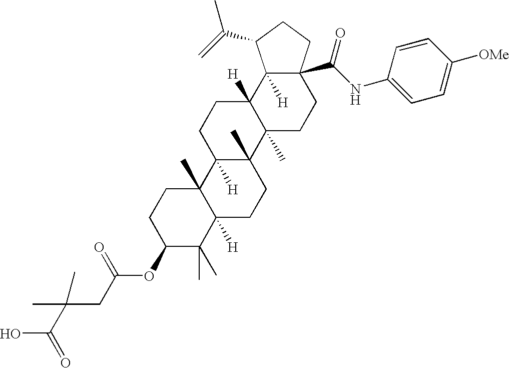 | 0.77 (s, 3 H, CH₃), 0.78 (s, 3 H, CH₃), 0.80 (s, 3 H, CH₃), 0.86 (s, 9 H, CH₃), 0.96 (s, 3 H, CH₃), 1.15 (s, 3 H, CH₃), 1.16 (s, 3 H, CH₃), 1.20-2.05 (m, 28 H), 2.30-2.40 (m, 1 H), 2.95-3.08 (m, 1 H), 3.71 (s, 3 H, CH₃), 4.36 (dd, 1 H, J = 11.4 Hz, J = 4.6 Hz, H-3), 4.55 (bs, 1 H, CH═), 4.68 (d, 1 H, J = 2.2 Hz, CH═), 6.85 (d, 2 H, J = 9.0 Hz, CH Arom), 7.45 (d, 2 H, J = 9.0 Hz, CH Arom), 9.29 (bs, 1 H, NH). 690.4727 (M + H)⁺. |
| 44 | 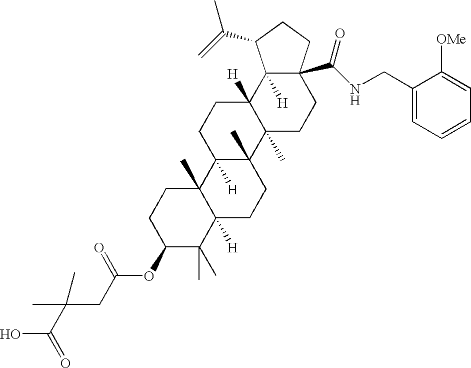 | 0.78 (s, 12 H, CH₃), 0.93 (s, 3 H, CH₃), 1.15 (s, 3 H, CH₃), 1.16 (s, 3 H, CH₃), 1.20-1.95 (m, 27 H), 2.15-2.25 (m, 1 H), 2.95-3.08 (m, 1 H), 3.79 (s, 3 H, CH₃O), 4.20 (d, 2 H, J = 5.9 Hz, CH₂N), 4.36 (dd, 1 H, J = 11.3 Hz, J = 4.7 Hz, H-3), 4.53 (bs, 1 H, CH═), 4.64 (d, 1 H, J = 2.5 Hz, CH═), 6.86 (dt, 1 H, J = 7.4 Hz, J = 1.0 Hz, CH Arom), 6.95 (d, 1 H, J = 7.6 Hz, CH Arom), 7.11 (d, 1 H, J = 7.4 Hz, CH Arom), 7.20 (dt, 1 H, J = 7.7 Hz, J = 1.7 Hz, CH Arom), 7.97 (t, 1 H, J = 5.9 Hz, NH), 12.18 (bs, 1 H, CO₂H). 704.4877 (M + H)⁺. |
| 45 | 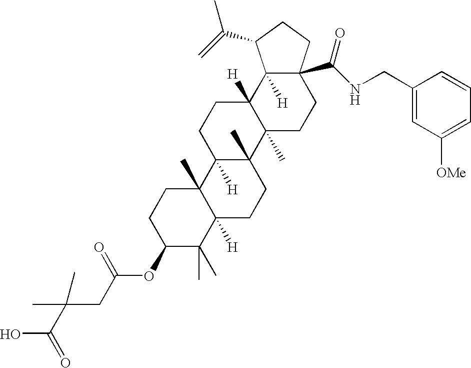 | 0.78 (s, 12 H, CH₃), 0.92 (s, 3 H, CH₃), 1.15 (s, 3 H, CH₃), 1.16 (s, 3 H, CH₃), 1.20-1.90 (m, 28 H), 2.15-2.25 (m, 1 H), 2.95-3.08 (m, 1 H), 3.71 (s, 3 H, CH₃O), 4.17 (dd, 1 H, J = 15.2 Hz, J = 6.0 Hz, CH₂N), 4.25 (dd, 1 H, J = 15.2 Hz, J = 6.1 Hz, CH₂N), 4.36 (dd, 1 H, J = 11.3 Hz, J = 4.7 Hz, H-3), 4.53 (bs, 1 H, CH═), 4.65 (d, 1 H, J = 1.8 Hz, CH═), 6.72-6.82 (m, 3 H, CH Arom), 7.19 (t, 1 H, J = 7.8 Hz, CH Arom), 8.16 (t, 1 H, J = 6.0 Hz, NH), 12.18 (bs, 1 H, CO₂H). 704.4870 (M + H)⁺. |

### General Procedure for Synthesizing Compound (46)

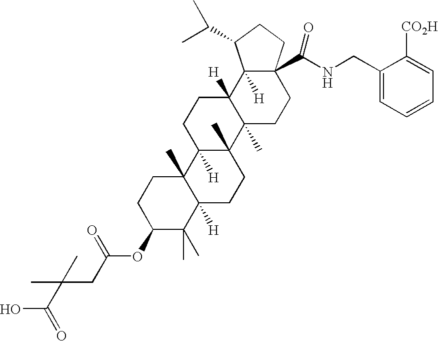

Compound 39 (0.112 g, 0.15 mmol) was suspended in glacial acetic acid (10 mL) and flushed with nitrogen. A catalytic amount of platinum (IV) oxide (0.012 g) was added. The reaction was placed under 15 psi of hydrogen gas overnight. The mixture was filtered through a pad of celite and the solvent was evaporated under reduced pressure. The crude material obtained was purified by HPLC. ¹H NMR (DMSO-d₆) δ 0.72 (s, 6H, CH₃), 0.785 (s, 12H, CH₃), 0.91 (s, 3H, CH₃), 1.16 (s, 3H, CH₃), 1.17 (s, 3H, CH₃), (m, 26H), 2.10-2.25 (m, 2H), 4.37 (dd, 1H, J=11.3 Hz, J=5.0 Hz, H-3), 4.53 (d, 2H, J=5.9 Hz, CH₂N), 7.30-7.38 (m, 2H, CH Arom), 7.49 (t, 1H, J=6.9 Hz, CH Arom), 7.85 (dd, 1H, J=7.6 Hz, J=1.4 Hz, CH Arom), 7.99 (t, 1H, J=5.9 Hz, NH), 12.25 (bs, 1H, CO₂H), 13.00 (bs, 1H, CO₂H).

LC-MS (ESI): 720.4837 (M+H)⁺.

### Example 2

### Determination of Antiviral Activity

The compounds of the invention can be tested in the following assays to detect antiviral activity and general toxicity.

### MT-4 Cytoprotection Assay

The HTLV-1 transformed T cell line, MT-4, is highly susceptible to HIV-1 infection. Anti-HIV-1 agents were evaluated in this target cell line by protection from the HIV-induced cytopathic effect. In this assay, viability of both HIV-1 and mock-infected cells was assessed in a calorimetric assay that monitors the ability of metabolically-active cells to reduce the tetrazolium salt WST-1. Cytoprotection by antiviral compounds is indicated by the positive readout of increased WST-1 cleavage.

Briefly, exponentially growing MT-4 cells were mock-infected or batch-infected with the HIV-1 laboratory strain, NL4-3, at a multiplicity of infection of 0.0005. Following a two hour infection, the cells were washed to remove unbound virus and plated in the presence of increasing concentrations of compound. After four days incubation, cytoprotection in the infected cells and compound toxicity in mock-infected cells were analyzed using the WST-1 assay.

### PBMC Drug Susceptibility Assay

Human peripheral blood mononuclear cells (PBMCs) were used to test compound antiviral activity as an indicator for clinical efficacy. PBMCs were isolated from two donors using a Ficoll-Hypaque density gradient, pooled and stimulated with PHA-L for three days. After stimulation, the cells were washed and maintained in culture medium containing IL-2. The stimulated cells were then mock-infected or batch-infected with the strain HIV-1^{IIIB} at MOI 0.01 for one hour. Cells (unwashed) were then plated in the presence of increasing concentrations of compound and incubated for seven days. The readout for virus replication in these cultures is the concentration of HIV-1 p24 in the supernatant because PBMCs generally do not succumb to HIV-induced cytopathic effects. Compound toxicity in mock-infected cells was analyzed using the WST-1 assay.

It was found that compounds of the invention have antiviral activity according to these assays. Compound 37 has an EC50 (concentration of compound that reduces the virus induced cytopathic effect by 50% (MT-4)(antiviral activity measure)) of about 126 nanomolar and a TC50 (TC50 is the concentration of compound that results in death of 50% of the host cells (toxicity measure)) of about 7.7 micromolar. Compound 39 has an EC50 of about 8.1 nanomolar and a TC50 of about 6.3 micromolar. Compound 36 has an EC50 of about 2.9 nanomolar and a TC50 of greater than 10 micromolar. Compound 42 has an EC50 of about 11 nanomolar and a TC50 of greater than 10 micromolar. Compound 30 has an EC50 of about 8.6 micromolar and a TC50 of greater than 10 micromolar.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be obvious that certain changes and modifications may be practiced within the scope of the appended claims.

1-23. (canceled)

24. A compound having a structure 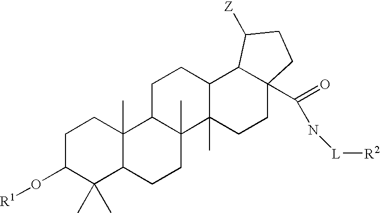
and pharmaceutically acceptable salts and stereoisomers thereof, wherein
L is —(CH₂)ₙ—, wherein n is an integer chosen from 0, 1, 2, and 3, and L can be substituted with alkyl;
R¹ is chosen from —C(═O)—(CH₂)ₘ—CH₃ and —C(═O)—(CH₂)ₘ—C(CH₃)₂—COOH, wherein m is an integer chosen from 0-10;
R² is heteroaryl optionally substituted with methyl, C-carboxy, hydroxy alkyl, alkoxy alkyl, C-carboxy alkyl, and aryl alkyl; and
Z is chosen from isopropyl and isopropenyl.

25. The compound according to claim 24, wherein R¹ is

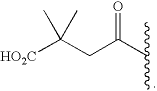

26. The compound according to claim 24, wherein Z is isopropenyl.

27. The compound according to claim 24, wherein
R¹ is 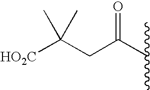
and
Z is isopropenyl.

28. A pharmaceutical composition comprising a compound according to claim 24, and a pharmaceutically acceptable carrier.

29. The pharmaceutical composition according to claim 28, further comprising an antiviral agent.

30. The pharmaceutical composition according to claim 29, wherein said antiviral agent is amantadine.

31. A method for inhibiting a retroviral infection in cells or tissue of an animal comprising administering an effective retroviral inhibiting amount of a compound according to claim 24.

32. The method according to claim 31, wherein said retroviral infection does not respond to other therapies.

33. A method for inhibiting a retroviral infection in cells or tissue of an animal comprising administering an effective retroviral inhibiting amount of a pharmaceutical composition according to claim 28.

34. The method according to claim 33, wherein the retroviral infection does not respond to other therapies.

35. The method according to claim 33, wherein said composition is administered to provide said compound in an amount ranging from about 0.01 μg/kg to about 100 mg/kg body weight.

36. The method according to claim 33, wherein said animal is a human.

37. A method of delaying the onset of HIV infection from a mother to a baby, comprising administering to said mother and/or said baby a therapeutically effective amount of a compound of claim 24, during pregnancy, delivery, or shortly thereafter.

38. A method of delaying the onset of HIV infection in an individual who has sex with an infected person, comprising administering a therapeutically effective amount of a compound of claim 24 to vaginal or other mucosa prior to having sex.

39. A compound having a structure 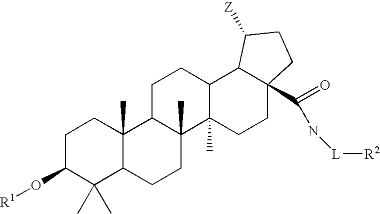
and pharmaceutically acceptable salts and stereoisomers thereof, wherein
L is —(CH₂)ₙ—, wherein n is an integer chosen from 0, 1, 2, and 3, and L can be substituted with alkyl;
R¹ is chosen from —C(═O)—(CH₂)ₘ—CH₃ and —C(═O)—(CH₂)ₘ—C(CH₃)₂—COOH, wherein m is an integer chosen from 0-10;
R² is heteroaryl optionally substituted with methyl, C-carboxy, hydroxy alkyl, alkoxy alkyl, C-carboxy alkyl, and aryl alkyl; and
Z is chosen from isopropyl and isopropenyl.

40. The compound according to claim 39, wherein R¹ is

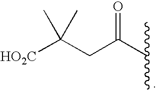

41. The compound according to claim 39, wherein Z is isopropenyl.

42. The compound according to claim 39, wherein
R¹ is 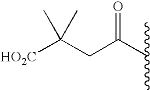
and
Z is isopropenyl.

43. A pharmaceutical composition comprising a compound according to claim 39, and a pharmaceutically acceptable carrier.

44. The pharmaceutical composition according to claim 43, further comprising an antiviral agent.

45. The pharmaceutical composition according to claim 44, wherein said antiviral agent is amantadine.

46. A method for inhibiting a retroviral infection in cells or tissue of an animal comprising administering an effective retroviral inhibiting amount of a compound according to claim 39.

47. The method according to claim 46, wherein said retroviral infection does not respond to other therapies.

48. A method for inhibiting a retroviral infection in cells or tissue of an animal comprising administering an effective retroviral inhibiting amount of a pharmaceutical composition according to claim 43.

49. The method according to claim 48, wherein the retroviral infection does not respond to other therapies.

50. The method according to claim 48, wherein said composition is administered to provide said compound in an amount ranging from about 0.01 μg/kg to about 100 mg/kg body weight.

51. The method according to claim 46, wherein said animal is a human.

52. A method of delaying the onset of HIV infection from a mother to a baby, comprising administering to said mother and/or said baby a therapeutically effective amount of a compound of claim 39, during pregnancy, delivery, or shortly thereafter.

53. A method of delaying the onset of HIV infection in an individual who has sex with an infected person, comprising administering a therapeutically effective amount of a compound of claim 39 to vaginal or other mucosa prior to having sex.
